(19) United States
(12) Patent Application Publication
(10) Pub. No.: US 2009/0036776 A1
(43) Pub. Date: Feb. 5, 2009

(54) Apparatus for measuring inner diameter of tubular body of living being

(75) Inventors: Hiroshi Masuda, Nagoya-shi (JP); Hitoshi Hirano, Nagoya-shi (JP); Hidenori Suzuki, Nagoya-shi (JP); Chikao Harada, Nagoya-shi (JP)

Correspondence Address: OLIFF & BERRIDGE, PLC, P.O. BOX 320850, ALEXANDRIA, VA, 22320-4850, US

(73) Assignee: UNEX CORPORATION, NAGOYA-SHI (JP)

(21) Appl. No.: 11/654,659

(22) Filed: Jan. 18, 2007

(30) Foreign Application Priority Data
Jan. 25, 2006 (JP) 2006-016151

(51) Int. Cl. A61B 8/00

(52) U.S. Cl. 600/449

(57) ABSTRACT

An apparatus for measuring an inner diameter of a tubular body present under a skin of a living being, by emitting an ultrasonic wave from an ultrasonic probe placed on the skin, the apparatus including a reflection-signal detecting portion which detects a reflection signal that is reflected from the tubular body when the ultrasonic wave is emitted from the ultrasonic probe; and an inner-diameter calculating portion which calculates the inner diameter of the tubular body, based on an interval between two groups of reflection waves which are contained by the detected reflection signal and which are reflected from two diametrically opposed portions of a wall of the tubular body, respectively.

The present application is based on Japanese Patent Application No. 2006-016151 filed on Jan. 25, 2006, the contents of which are incorporated herein by reference.

### BACKGROUND OF THE INVENTION

1. Field of the Invention

The present invention relates to an apparatus for measuring an inner diameter of a tubular body or tissue (e.g., an artery, a vein, or a lymph duct) of a living being, located under the skin, by emitting, from an emission surface of an ultrasonic probe placed on the skin, an ultrasonic wave toward the tubular body.

2. Related Art Statement

For example, Japanese Patent Application Publication No. 2003-180690 discloses an apparatus for measuring a diameter of an artery as one of tubular bodies present in a living being. Since a function of an endothelium of the artery that is indicated by, e.g., a rate of increase of a diameter thereof at the time of reactive hyperemia is related to a degree of arteriosclerosis, there is a need to provide a non-invasive and simple method of measuring the diameter of artery so as to be able to make an early diagnosis of arteriosclerosis.

In the above-indicated artery-diameter measuring method, first, an ultrasonic probe is placed on an upper arm of the living subject, so as to emit an ultrasonic wave toward the artery located under the skin, and obtain, based on an ultrasonic reflection signal reflected from the artery, an ultrasonic cross-section image of the artery. Then, based on a variable-density signal representing the ultrasonic cross-section image, respective positions of two diametrically opposed portions of the wall of the artery are determined, and finally a diameter of the artery is determined from a distance between the two wall portions. As one of evaluation indexes that are used in evaluating a degree of arteriosclerosis by utilizing the function of endothelium of artery, there is known an FMD (flow-mediated dilation) value [=100×(dmax−d)/d] that is detected in such a manner that first an image of the artery before the flow of blood therein is stopped and an image of the artery after the stopping of the blood flow is ended are obtained and then an FMD value, i.e., a rate of increase of a diameter, dmax, of the artery at the time of dilation, from a diameter, d, of the same at the time of constriction is calculated.

### SUMMARY OF THE INVENTION

Meanwhile, in the above-indicated artery-diameter measuring apparatus, a degree of arteriosclerosis is evaluated based on a small change of diameter of an artery. Therefore, it is needed to measure accurately the diameter of the artery. However, the conventional artery-diameter measuring apparatus determines the diameter of the artery, based on the variable-density signal representing the ultrasonic cross-section image of the artery. The variable-density signal may be obtained in such a manner that first the ultrasonic reflection signal is converted, by being rectified and squared, into the power of the signal, subsequently the power of the signal is converted, by being smoothed in an envelope-smoothing process, into a smoothed signal that smoothly changes, and then the smoothed signal is converted into the stepwise variable density signal. The thus detected variable density signal can be advantageously used, as it is, to scan an image screen and thereby display a two-dimensional ultrasonic cross-section image of the artery.

However, the variable density signal has such a drawback that a portion of the signal that is detected in a certain time duration starting with the time of reflection at an interface of the wall of the artery, i.e., an interface where the impedance of ultrasonic-wave transmitting medium changes, indicates a uniform density. Therefore, if the diameter of the artery is measured as the interval between respective portions of the artery each of which corresponds to a uniform density of the signal that indicates the presence of wall of the artery, then a diameter of a central, black area of the cross-section image of the artery is measured as the inner diameter of the artery. However, the thus measured inner diameter tends to be smaller than an actual inner diameter, i.e., tends to be more or less inaccurate. This is also the case with other sorts of tubular bodies or tissues, such as a vein or a lymph duct, present under the skin.

It is therefore an object of the present invention to provide a tubular-body-inner-diameter measuring apparatus that can accurately measure an inner diameter of a tubular body present under skin of a living being.

The above-indicated object has been achieved by the present invention. According to a first mode of the present invention, there is provided an apparatus for measuring an inner diameter of a tubular body present under a skin of a living being, by emitting an ultrasonic wave from an ultrasonic probe placed on the skin, the apparatus comprising a reflection-signal detecting portion which detects a reflection signal that is reflected from the tubular body when the ultrasonic wave is emitted from the ultrasonic probe; and an inner-diameter calculating portion which calculates the inner diameter of the tubular body, based on an interval between two groups of reflection waves which are contained by the detected reflection signal and which are reflected from two diametrically opposed portions of a wall of the tubular body, respectively.

The present tubular-body-inner-diameter measuring apparatus detects the reflection signal reflected from the tubular body when the ultrasonic wave is emitted from the ultrasonic probe toward the tubular body, determines the interval between the two groups of reflection waves that are contained by the detected reflection signal and that are respectively reflected from the two diametrically opposed portions of the wall of the tubular body, and directly calculates, based on the determined interval, the inner diameter of the tubular body. The reflection signal contains substantially all fine amplitudes thereof (i.e., strong and weak amplitudes), and accordingly contains important time-related information, that would be lost if the detected reflection signal is subjected to the envelope-smoothing process that may be used to produce an ultrasonic cross-section image of the tubular body. Since the inner diameter of the tubular body is calculated based on the interval between the two groups of fine reflection waves, the thus calculated inner diameter enjoys a high accuracy.

According to a second mode of the present invention, the apparatus according to the first mode further comprises a signal rectifying portion which rectifies the detected reflection signal containing the two groups of reflection waves reflected from the tubular body; and a peak-time-signal producing portion which produces, from the rectified reflection signal containing the two groups of reflection waves, a peak-time signal which represents a magnitude and a time position of a peak of each of respective waveforms of the reflection waves of the two groups, wherein the inner-diameter calculating portion calculates the inner diameter of the tubular body, based on an interval between two groups of peak-time-signal waves which are contained by the produced peak-time signal and which correspond to the two groups of reflection waves, respectively.

In the present tubular-body-inner-diameter measuring apparatus, the peak-time-signal producing portion produces, from the rectified reflection signal containing the two groups of reflection waves reflected from the tubular body, the peak-time signal which indicates the magnitude, and the occurrence time, of the peak of waveform of each of the reflection waves of the two groups, and the inner-diameter calculating portion calculates the inner diameter of the tubular body, based on the interval between the two groups of peak-time-signal waves that are contained by the peak-time signal and that correspond to the two groups of reflection waves, respectively. Thus, an accurate inner diameter can be measured. In addition, since the peak-time signal consists of sets of data each set of which indicates the magnitude, and time, of the peak of waveform of a corresponding one of the reflection waves, an amount of data as the peak-time signal only ranges from one third to one fifth of an amount of data as the detected reflection signal, i.e., the sampled time-discrete reflection signal. This contributes to reducing the capacity of a memory(s) or a hard disc(s) employed by the present apparatus, and decreasing a signal-processing load of the same.

According to a third mode of the present invention, the apparatus according to the second mode further comprises a measurement-position determining portion which determines two measurement positions to measure the interval between the two groups of peak-time-signal waves which are contained by the peak-time signal and which correspond to the two groups of reflection waves, respectively, wherein the inner-diameter calculating portion calculates the inner diameter of the tubular body, based on an interval between the two measurement positions determined by the measurement-position determining portion.

In the present tubular-body-inner-diameter measuring apparatus, the measurement-position determining portion determines the two measurement positions to measure the interval between the two groups of peak-time-signal waves that are contained by the peak-time signal, and the inner-diameter calculating portion calculates the inner diameter of the tubular body, based on the interval between the two measurement positions determined by the measurement-position determining portion. Since the inner diameter of the tubular body is calculated based on the interval between the two measurement positions determined by the measurement-position determining portion, the thus calculated inner diameter enjoys an improved accuracy.

According to a fourth mode of the present invention, in the apparatus according to the third mode, the measurement-position determining portion determines, as the two measurement positions to measure the interval between the two groups of peak-time-signal waves which are contained by the peak-time signal and which correspond to the two groups of reflection waves, respectively, a terminal end of a first series of waves located in a terminal portion of a first group of peak-time-signal waves of the two groups of peak-time-signal waves, and an initial end of a second series of waves located in an initial portion of a second group of peak-time-signal waves of the two groups of peak-time-signal waves.

In the present tubular-body-inner-diameter measuring apparatus, the measurement-position determining portion determines, as the two measurement positions to measure the interval between the two groups of peak-time-signal waves that correspond to the two groups of reflection waves reflected from the two diametrically opposed portions of the wall of the tubular body, the terminal end of the first series of waves located in the terminal portion of the first group of peak-time-signal waves, and the initial end of the second series of waves located in the initial portion of the second group of peak-time-signal waves. Therefore, the inner-diameter calculating portion calculates the inner diameter of the tubular body, based on the interval between the terminal end of the first series of waves located in the terminal portion of the first group of peak-time-signal waves, and the initial end of the second series of waves located in the initial portion of the second group of peak-time-signal waves. Thus, a more accurate inner diameter of the tubular body can be measured while an endothelium of the body is taken into account.

According to a fifth mode of the present invention, the apparatus according to any of the second to fourth modes further comprises a display control portion which controls an image display device to display a waveform of the peak-time signal containing the two groups of peak-time-signal waves.

In the present tubular-body-inner-diameter measuring apparatus, the display control portion controls the image display device to display the waveform of the peak-time signal containing the two groups of peak-time-signal waves. Therefore, an observer such as a doctor can observe, on the image display device, the inner diameter of the tubular body that corresponds to the interval between the two groups of peak-time-signal waves.

According to a sixth mode of the present invention, the apparatus according to the fifth mode further comprises an ultrasonic-cross-section-image producing portion which produces, based on the detected reflection signal reflected from the tubular body, an ultrasonic cross-section image of the tubular body present under the skin, wherein the display control portion controls the image display device to display the waveform of the peak-time signal and the ultrasonic cross-section image, side by side, such that a time axis of the waveform of the peak-time signal and a direction of depth of the cross-section image are parallel to each other.

In the present tubular-body-inner-diameter measuring apparatus, the ultrasonic-cross-section-image producing portion produces, based on the detected reflection signal reflected from the tubular body, the cross-section image of the tubular body present under the skin of the living being, and the display control portion controls the image display device to display the waveform of the peak-time signal and the cross-section image, side by side, such that the time axis of the waveform of the peak-time signal and the direction of depth of the cross-section image are parallel to each other. Therefore, an observer can observe, on the image display device, the inner diameter of the tubular body in the state in which the waveform of the peak-time signal and the ultrasonic cross-section image can be compared with each other.

According to a seventh mode of the present invention, in the apparatus according to the sixth mode, the display control portion controls the image display device to display the waveform of the peak-time signal and the cross-section image, such that the interval between the two groups of peak-time-signal waves contained by the peak-time signal, and the inner diameter of the wall of the tubular body, contained by the ultrasonic cross-section image, have a same size and a same position with respect to the time axis and the direction of depth.

In the present tubular-body-inner-diameter measuring apparatus, the display control portion controls the image display device to display the waveform of the peak-time signal and the cross-section image, such that the interval between the two groups of peak-time-signal waves contained by the peak-time signal, and the inner diameter of the wall of the tubular body, contained by the cross-section image, have a same size and have a same position on the image display device. Therefore, an observer can observe, on the image display device, the interval between the two groups of peak-time-signal waves contained by the peak-time signal, and an inner lumen of the tubular body contained by the cross-section image, in the state in which the interval and the lumen of the tubular body are arranged side by side so as to be comparable with each other, and have the same size and the same height position. Thus, the observer can more easily observe the inner diameter of the tubular body of the living being.

According to an eighth mode of the present invention, the apparatus according to the first mode further comprises a measurement-position determining portion which determines two measurement positions to measure the interval between the two groups of reflection waves which are contained by the reflection signal detected by the reflection-signal detecting portion and which are reflected from the two diametrically opposed portions of the wall of the tubular body, respectively, wherein the inner-diameter calculating portion calculates the inner diameter of the tubular body, based on an interval between the two measurement positions determined by the measurement-position determining portion.

In the present tubular-body-inner-diameter measuring apparatus, the measurement-position determining portion determines the two measurement positions to measure the interval between the two groups of reflection waves that are contained by the reflection signal detected by the reflection-signal detecting portion and that are reflected from the wall of the tubular body, and the inner-diameter calculating portion calculates the inner diameter of the tubular body, based on the interval between the two measurement positions determined by the measurement-position determining portion. Since the inner diameter of the tubular body is calculated based on the interval between the two measurement positions determined by the measurement-position determining portion, the thus calculated inner diameter enjoys an improved accuracy.

According to a ninth mode of the present invention, in the apparatus according to the eighth mode, the measurement-position determining portion determines, as the two measurement positions to measure the interval between the two groups of reflection waves contained by the detected reflection signal reflected from the tubular body, a terminal end of a first series of waves located in a terminal portion of a first group of reflection waves of the two groups of reflection waves, and an initial end of a second series of waves located in an initial portion of a second group of reflection waves of the two groups of reflection waves.

In the present tubular-body-inner-diameter measuring apparatus, the measurement-position determining portion determines, as the two measurement positions to measure the interval between the two groups of reflection waves contained by the reflection signal reflected from the tubular body, the terminal end of the first series of waves located in the terminal portion of the first group of reflection waves of the two groups of reflection waves, and the initial end of the second series of waves located in the initial portion of the second group of reflection waves of the two groups of reflection waves. Therefore, the inner-diameter calculating portion calculates the inner diameter of the tubular body, based on the interval between the terminal end of the first series of waves located in the terminal portion of the first group of reflection waves, and the initial end of the second series of waves located in the initial portion of the second group of reflection waves. Thus, a more accurate inner diameter of the tubular body can be measured while an endothelium of the tubular body is taken into account.

According to a tenth mode of the present invention, the apparatus according to the eighth or ninth mode further comprises a display control portion which controls an image display device to display a waveform of the detected reflection signal.

In the present tubular-body-inner-diameter measuring apparatus, the display control portion controls the image display device to display the waveform of the reflection signal including the two groups of reflection waves. Therefore, an observer can observe, on the image display device, the inner diameter of the tubular body that corresponds to the interval between the two groups of reflection waves.

According to an eleventh mode of the present invention, the apparatus according to the tenth mode further comprises an ultrasonic-cross-section-image producing portion which produces, based on the detected reflection signal reflected from the tubular body, an ultrasonic cross-section image of the tubular body present under the skin, wherein the display control portion controls the image display device to display the waveform of the detected reflection signal and the ultrasonic cross-section image, side by side, such that a time axis of the waveform of the reflection signal and a direction of depth of the cross-section image are parallel to each other.

In the present tubular-body-inner-diameter measuring apparatus, the ultrasonic-cross-section-image producing portion produces, based on the reflection signal reflected from the tubular body, the cross-section image of the tubular body present under the skin, and the display control portion controls the image display device to display the waveform of the reflection signal and the cross-section image, side by side, such that the time axis of the waveform of the reflection signal and the direction of depth of the cross-section image are parallel to each other. Therefore, an observer can observe, on the image display device, the inner diameter of the tubular body in the state in which the waveform of the reflection signal and the ultrasonic cross-section image can be compared with each other.

According to a twelfth mode of the present invention, in the apparatus according to the eleventh mode, the display control portion controls the image display device to display the waveform of the detected reflection signal and the ultrasonic cross-section image, such that the interval between the two groups of reflection waves contained by the detected reflection signal, and the inner diameter of the wall of the tubular body, contained by the cross-section image, have a same size and have a same position with respect to the time axis and the direction of depth.

In the present tubular-body-inner-diameter measuring apparatus, the display control portion controls the image display device to display the waveform of the reflection signal and the cross-section image, such that the interval between the two groups of reflection waves contained by the reflection signal and the inner diameter of the wall of the tubular body, contained by the cross-section image, have the same size and have the same position on the image display device. Therefore, an observer can observe, on the image display device, the interval between the two groups of reflection waves contained by the reflection signal, and an inner lumen of the tubular body contained by the cross-section image, in the state in which the interval and the lumen of the tubular body are arranged side by side so as to be comparable with each other, and have the same size and the same height position. Thus, the observer can more easily observe the inner diameter of the tubular body.

According to a thirteenth mode of the present invention, in the apparatus according to any of the first to twelfth modes, the tubular body is selected from the group consisting of an artery, a vein, and a lymph duct.

In the present tubular-body-inner-diameter measuring apparatus, the tubular body is selected from the group consisting of the artery, the vein, and the lymph duct present in the living being. Therefore, an inner diameter of the artery can be accurately detected while an endothelium of the artery is taken into account; and an inner diameter of the vein or the lymph duct can be accurately detected.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a schematic view for explaining a construction of a blood-vessel-endothelium-function inspecting apparatus as a first embodiment of the present invention;

FIG. 2 is an enlarged view showing a free end portion of a sensor holding apparatus of the inspecting apparatus of FIG. 1, the free end portion supporting a universal joint that holds an ultrasonic probe, and a stopper device to stop or inhibit the rotation of the universal joint;

FIG. 3 is a diagrammatic view showing an electric arrangement including an ultrasonic array provided at an end portion of the ultrasonic probe of FIG. 2, an ultrasonic-wave control circuit, and an electronic control device;

FIG. 4 is a diagrammatic view for explaining relevant control functions of the control device of FIG. 3;

FIG. 5 is a graph showing a portion of a reflection signal, SR, detected by a reflection-signal detecting portion shown in FIG. 4;

FIG. 6 is a graph showing a portion of the reflection signal rectified by a signal rectifying portion shown in FIG. 4;

FIG. 7 is a graph showing a portion of a peak-time signal, SP, produced by a peak-time-signal producing portion shown in FIG. 4;

FIG. 8 is a graph showing an example of a combination of an ultrasonic cross-section image, MG, and a peak-time signal SP that is displayed under control of a display control portion shown in FIG. 4;

FIG. 9 is a graph showing another example of a combination of an ultrasonic cross-section image MG and a peak-time signal SP that is displayed under control of the display control portion;

FIG. 10 is a graph showing another example of a combination of an ultrasonic cross-section image MG and a peak-time signal SP that is displayed under control of the display control portion;

FIG. 11 is a graph showing another example of a combination of an ultrasonic cross-section image MG and a peak-time signal SP that is displayed under control of the display control portion;

FIG. 12 is a graph showing another example of a combination of an ultrasonic cross-section image MG and a peak-time signal SP that is displayed under control of the display control portion;

FIG. 13 is a graph showing a time-wise change of an inner diameter of an artery, and a % FMD value as an evaluation index that is used in evaluating a function of an endothelium of the artery;

FIG. 14 is a flow chart representing a control operation of the control device of FIG. 3;

FIG. 15 is a diagrammatic view corresponding to FIG. 4, for explaining relevant control functions of another electronic control device of another blood-vessel-endothelium-function inspecting apparatus as a second embodiment of the present invention;

FIG. 16 is a graph showing an example of a combination of an ultrasonic cross-section image MG and a reflection signal SR that is displayed under control of a display control portion shown in FIG. 15;

FIG. 17 is a graph showing another example of a combination of an ultrasonic cross-section image MG and a reflection signal SR that is displayed under control of the displaying portion of FIG. 15;

FIG. 18 is a flow chart representing a control operation of the control device shown in FIG. 15;

FIG. 19 is a graph showing an example of a combination of an ultrasonic cross-section image MG and a peak-time signal SP that is displayed under control of a display control portion of another electronic control device employed by another inner-diameter measuring apparatus as a third embodiment of the present invention that has a construction similar to that of the blood-vessel-endothelium-function inspecting apparatus of FIG. 4 and measures an inner diameter of a vein or a lymphatic vessel; and

FIG. 20 is a graph showing another example corresponding to the example shown in FIG. 19, wherein a reflection signal reflected from a boundary layer where ultrasonic-wave impedance significantly changes, continues for a long time duration.

### DETAILED DESCRIPTION OF PREFERRED EMBODIMENTS

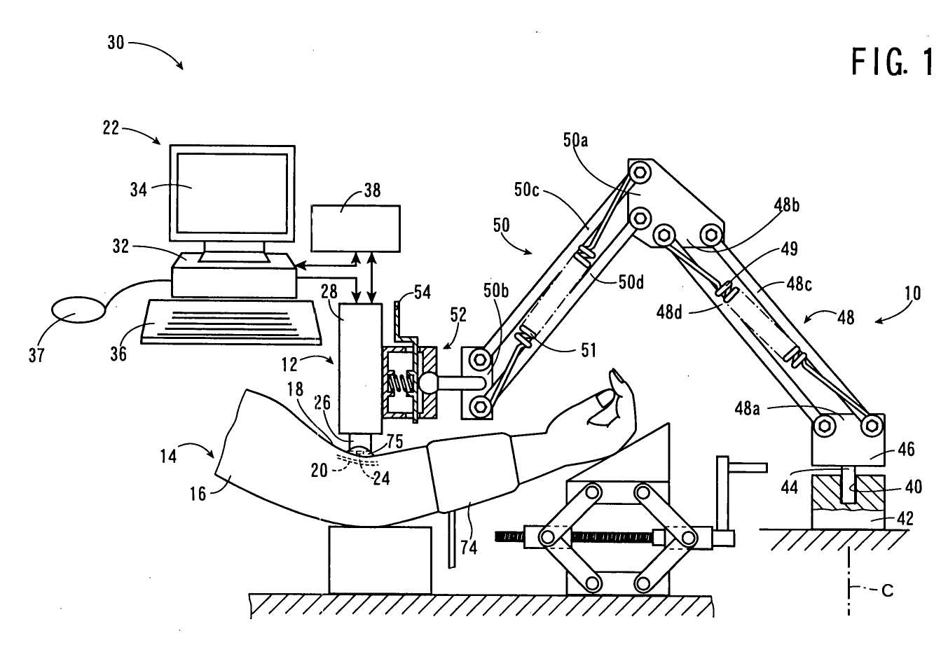

Hereinafter, there will be described preferred embodiments of the present invention in detail by reference to the drawings. FIG. 1 is a front view for explaining a blood-vessel-endothelium-function inspecting apparatus 30 including a blood-vessel-image displaying device 22 which includes an ultrasonic probe 12, and a sensor holding device 10 that holds the ultrasonic probe 12, and which displays, using the ultrasonic probe 12 held on a skin 18 of an upper arm 16 of a living being 14 (e.g., a living person) as an object to be measured, a transverse-cross-section image (i.e., a short-axis image) and/or a longitudinal-cross-section image (i.e., a long-axis image) of a blood vessel (e.g., an artery) 20 located under the skin 18. The blood-vessel-endothelium-function inspecting apparatus 30 measures, for evaluating a function of an endothelium of the blood vessel 20, a rate of change of a diameter of the blood vessel 20 that represents a % FMD (flow-mediated dilation) value following postischemia reactive hyperemia, in such a manner that, first, flow of blood in the blood vessel 20 is stopped for an appropriate time duration by inflation of a cuff 74 that is wound around a forearm of the living being 14, then the stopping of the blood flow is ended by deflation of the cuff 74, and finally a change of diameter of the blood vessel 20 after the deflation of the cuff 74 is detected. Therefore, the blood-vessel-endothelium-function inspecting apparatus 30 also functions as an apparatus for measuring an inner diameter of a tubular body of the living being 14.

The ultrasonic probe 12 also functions as a blood-vessel-diameter sensor, and has a free-end portion 24 supporting a large number of ultrasonic transducers, E, each of which is constituted by, e.g., piezoelectric ceramics and which are arranged in one array 24a (or two arrays parallel to each other); a multiple-axis driving or positioning device 26; and a main frame 28 that supports the free-end portion 24 via the multiple-axis positioning device 26.

The blood-vessel-endothelium-function inspecting apparatus 30 further includes an electronic control device 32 that is constituted by a so-called microcomputer; an image display device 34 that is used for monitoring of the inner diameter of the blood vessel 20; a keyboard 36 and a mouse 37 each as an input device; and an ultrasonic-wave control circuit 38. The control device 32 controls the ultrasonic-wave control circuit 38 to supply drive signals to the ultrasonic array 24a provided in the free-end portion 24 of the ultrasonic probe 12, so that the ultrasonic array 24a emits ultrasonic waves, receives the ultrasonic waves reflected from the tissue (e.g., the blood vessel 20) located under the skin 18, and produces a reflected-ultrasonic-wave signal (hereinafter, referred to as the “reflection signal”) SR. The control device 32 receives the reflection signal SR from the ultrasonic array 24a, processes the thus received reflection signal SR, produces an ultrasonic cross-section image of the tissue under the skin 18, and controls the image display device 34 to display the thus produced ultrasonic cross-section image. An outer surface of the free-end portion 24 corresponds to an emission surface, S, from which the ultrasonic array 24a emits the ultrasonic waves. When the control device 32 produces the transverse-cross-section image (i.e., the short-axis image) of the blood vessel 20, the control device 32 controls the three-axis positioning device 26 to position the ultrasonic array 24a of the free-end portion 24 relative to the blood vessel 20, such that the ultrasonic array 24a extends in a direction perpendicular to the blood vessel 20; and when the control device 32 produces the longitudinal-cross-section image (i.e., the long-axis image) of the blood vessel 20, the control device 32 controls the three-axis positioning device 26 to position the ultrasonic array 24a relative to the blood vessel 20, such that the ultrasonic array 24a extends in a direction parallel to the blood vessel 20.

The ultrasonic-wave control circuit 38 carries out, according to a command supplied from the control device 32, a beam-forming operation in which a predetermined number of ultrasonic transducers starting with one of opposite ends of the ultrasonic array 24a are simultaneously driven such that each of the transducers generates an ultrasonic wave having a frequency of about 10 MHz with a predetermined phase difference from the phase of the ultrasonic wave generated by each of the two transducers located adjacent the each transducer on either side of the same. While the predetermined number of transducers are shifted, one transducer by one, in a direction from the above-indicated one end of the ultrasonic array 24a to the other end thereof, the ultrasonic array 24a sequentially emits, toward the blood vessel 20, respective ultrasonic beams each of which is convergent with respect to the direction of extension of the ultrasonic array 24a. Each time the ultrasonic array 24a emits the ultrasonic beam, it receives the ultrasonic beam reflected from the wall of the blood vessel 20, and inputs an electric signal representing the received, reflected ultrasonic beam, to the control device 32. The outer surface of the free-end portion 24 in which the ultrasonic array 24a is provided is covered with an acoustic lens (not shown) that causes each ultrasonic beam to converge with respect to a direction perpendicular to the direction of extension of the ultrasonic array 24a.

The electronic control device 32 synthesizes or produces, based on the reflected ultrasonic beams detected by the ultrasonic array 24a, an ultrasonic transverse-cross-section image (i.e., a short-axis image) of the blood vessel 20 located under the skin 18, and/or an ultrasonic longitudinal-cross-section image (i.e., a long-axis image) of the blood vessel 20, and controls the image display device 34 to display the thus produced ultrasonic cross-section image(s) of the blood vessel 20. In addition, the control device 32 calculates, from the produced image(s) of the blood vessel 20, an inner diameter of the same 20, i.e., an inner diameter of an endothelium (i.e., an inner layer (tunica intima)) of the same 20. Moreover, for the purpose of evaluating a function of the endothelium of the blood vessel 20, the control device 32 calculates a rate of change (%) [=100×(dₘₐₓ−d)/d, where d is an inner diameter of the endothelium of the vessel 20 when the living being 14 is at rest; and dₘₐₓ is a maximum inner diameter of the endothelium of the vessel 20 after the flow of blood is resumed] of the inner diameter of the endothelium of the vessel 20 that represents a % FMD (flow-mediated dilation) value following postischemia reactive hyperemia. Based on the calculated inner diameter of endothelium of the blood vessel 20, the control device 32 can calculate an accurate cross-section area of an inner lumen of the blood vessel 20 through which blood flows. In addition, the control device 32 can calculate an accurate flow rate of the blood based on a flow velocity of the blood detected by, e.g., an ultrasonic Doppler device (not shown).

The ultrasonic probe 12 is held by the sensor holding apparatus 10, such that the probe 12 can take a desirable posture and can touch, at a desirable or predetermined position in a three-dimensional space, the skin 18 of the upper arm 16 of the living being 14, without changing a shape of the wall of blood vessel 20 located under the skin 18. Usually, a well-known coupling agent 75 such as a jelly is interposed between the skin 18 and the outer surface of the free end portion 24 of the ultrasonic probe 12, for the purpose of preventing the attenuation of ultrasonic waves, and/or the reflection or scattering thereof at the interface of the two elements 18, 24, and thereby obtaining clear ultrasonic images. The jelly 75 may be a gel of a hydrophilic polymer that contains water at a high proportion and has an intrinsic impedance [=(sound speed)×(density)] sufficiently higher than that of air, and accordingly effectively restrains the attenuation of ultrasonic-wave signals transmitted and received. The jelly 75 is, e.g., agar, but it may be replaced with a water bag, i.e., a water packed in a resin-based bag; olive oil; or glycerin.

The sensor holding apparatus 10 is fixed in position to a support member such as a desk or a seat. More specifically described, the sensor holding apparatus 10 includes a base member 42 having a fitting hole 40 extending along a vertical axis line, C; and a rotatable member 46 that has a fitting axis portion 44 that fits in the fitting hole 40 such that the axis portion 44 is rotatable relative thereto, so that the rotatable member 46 is rotatable about the vertical axis line C relative to the base member 42. The sensor holding apparatus 10 additionally includes a first link device 48 that is constituted by four links 48a, 48b, 48c, 48d including a horizontal, first stationary link 48a fixed to (i.e., integral with) the rotatable member 46; a second link device 50 that is constituted by four links 50a, 50b, 50c, 50d including a vertical, second stationary link 50a fixed to (i.e., integral with) an end portion of the first link device 48; a universal joint 52 that is fixed to an end portion of the second link device 50, connects the ultrasonic probe 12 to the same 50, and supports the probe 12 such that the probe 12 is universally rotatable; and a stopper device 56 that includes an operable lever 54 and that fixes the universal joint 52 while the lever 54 is not operated by an operator, and releases the fixation of the joint 52, i.e., permits the universal rotation of the joint 52 while the lever 54 is operated by the operator.

The first link device 48 includes the first stationary link 48a; a first movable link 48b extending parallel to the first stationary link 48a; and two first pivotable links 48c, 48d which extend parallel to each other and each of which is pivotably connected, at two opposite ends thereof, to the first stationary link 48a and the first movable link 48b, respectively, so that the first stationary link 48a, the first movable link 48b, and the two first pivotable links 48c, 48d cooperate with each other to define a parallelogram. The first stationary link 48a is fixed to the rotatable member 46 such that the first movable link 48b is movable in a plane containing the vertical axis line C. In association with the first link device 48, there is provided a first coil spring 49 functioning as a first biasing device that produces a thrust having a directional component resisting a load applied to the first movable link 48b. The first coil spring 49 is connected at one end thereof to a connection point where one first pivotable link 48c and the first stationary link 48a are connected to each other, and is connected at the other end thereof to a connection point where the other first pivotable link 48d and the first movable link 48b are connected to each other, such that a moment produced by the first coil spring 49 in a direction to move the first movable link 48b upward, and a moment produced by the load applied to the first movable link 48b in a direction to move the same 48b downward are substantially cancelled by each other.

The second link device 50 includes a pair of second pivotable links 50c, 50d that extend parallel to each other; and the second stationary link 50a and a second movable link 50b which extend parallel to each other and each of which is pivotably connected, at two opposite ends thereof, to the two second pivotable links 50c, 50d, respectively, so that the second stationary link 50a, the second movable link 50b, and the two second pivotable links 50c, 50d cooperate with each other to define a parallelogram. The second stationary link 50a is fixed to the first movable link 48b such that the second stationary link 50a extends in a direction substantially perpendicular to the first stationary link 48a, and such that the second movable link 50b is movable in the plane containing the vertical axis line C. In association with the second link device 50, there is provided a second coil spring 51 functioning as a second biasing device that produces a thrust having a directional component resisting a load applied to the second movable link 50b. The second coil spring 51 is connected at one end thereof to a connection point where one second pivotable link 50c and the second stationary link 50a are connected to each other, and is connected at the other end thereof to a connection point where the other second pivotable link 50d and the second movable link 50b are connected to each other, such that a moment produced by the second coil spring 51 in a direction to move the second movable link 50b upward, and a moment produced by the load applied to the second movable link 50b in a direction to move the same 50b downward are substantially cancelled by each other. Owing to the respective moment-canceling actions of the first and second coil springs 49, 51, the sensor holding apparatus 10 can hold the ultrasonic probe 12 such that the probe 12 is stopped at any desirable position, or is slowly moved downward, in the three-dimensional space, and such that the outer surface of the free end portion 24 of the probe 12 lightly touches the skin 18 of the living being 14 without deforming the wall of blood vessel 20 and closely contacts the same 18 via the coupling agent 75 such as the jelly.

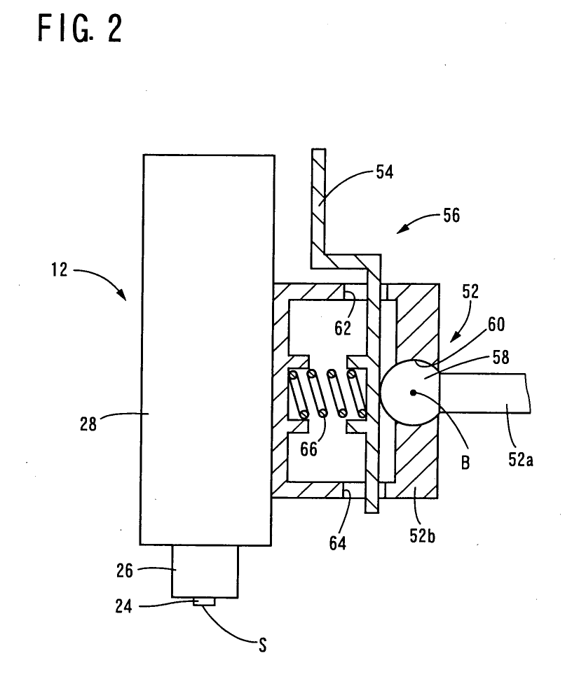

FIG. 2 is an enlarged view of the universal joint 52 and the stopper device 56. As shown in the figure, the universal joint 52 includes a first connection member 52a having a base end portion fixed to the second movable link 50b, and a free end portion 58 having a spherical shape; and a second connection member 52b that has a fitting hole 60 in which the spherical end portion 58 of the first connection member 52a slideably fits, and that is connected to the spherical end portion 58 such that the second connection member 52b is universally rotatable about a center, B, of the spherical portion 58. The second connection member 52b has two guide holes 62, 64 that cooperate with each other to guide the operable lever 54 of the stopper device 56 such that the operable lever 54 is movable toward, and away from, the spherical end portion 58 of the first connection member 52a.

The stopper device 56 includes, in addition to the operable lever 54, a pressing spring 66 that presses the operable lever 54 against the spherical end portion 58 of the first connection member 52a. In a usual state in which the operable lever 54 is not in use, the pressing spring 66 presses the operable lever 54 against the spherical portion 58, so as to inhibit the rotation of the universal joint 52 and thereby fix the same 52. However, when the operable lever 54 is used or operated by the operator against the biasing force of the pressing spring 66, and is moved away from the spherical portion 58, the fixation of the universal joint 52 is released and the universal rotation of the same 52 is permitted. Thus, the ultrasonic probe 12 is allowed to take a desirable posture.

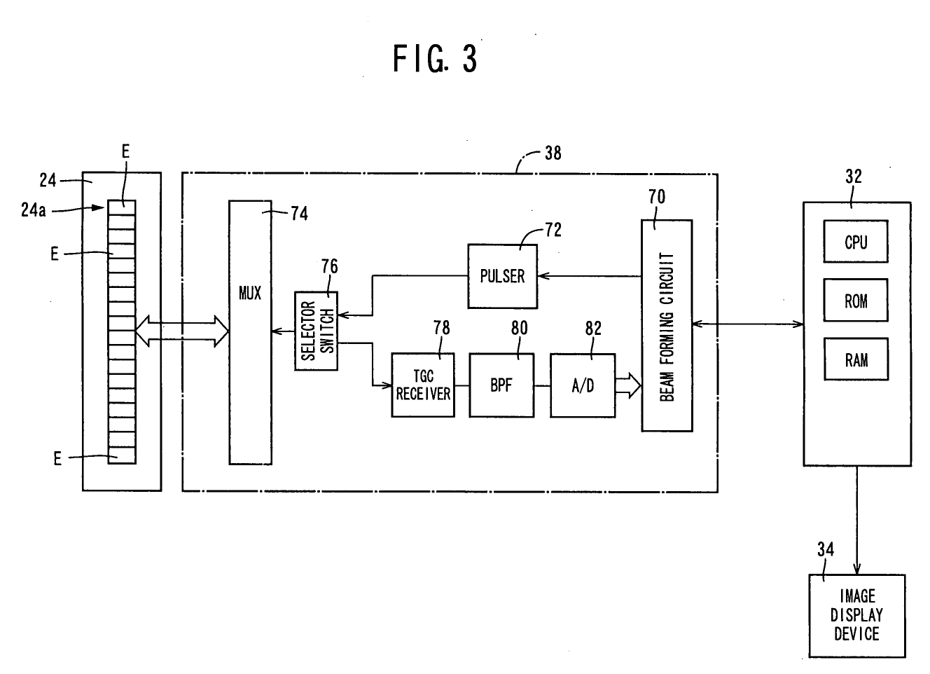

FIG. 3 is a diagrammatic view showing, in detail, an electrical arrangement including the ultrasonic array 24a provided in the free-end portion 24 of the ultrasonic probe 12, the ultrasonic-wave control circuit 38, and the electronic control device 32. In FIG. 3, the ultrasonic-wave control circuit 38 carries out, according to the command supplied from the control device 32, the beam-forming operation to emit sequentially the convergent ultrasonic beams toward the blood vessel 20 running in the direction perpendicular to the direction of extension of the ultrasonic array 24a. More specifically described, the ultrasonic array 24a consists of the large number of ultrasonic transducers (i.e., piezoelectric elements) E that are arranged in the direction intersecting the blood vessel 20, and a predetermined number of transducers (e.g., from 16 to 32 transducers) starting with one of the opposite ends of the ultrasonic array 24a are grouped and are simultaneously driven such that each of those transducers emits an ultrasonic wave, in a time duration equal to one period thereof, that has a frequency of about 10 MHz with a predetermined phase difference from the phase of the ultrasonic wave emitted by each of the two transducers located adjacent the each transducer on either side of the same. While the predetermined number of transducers are shifted, one transducer by one, in the direction from the above-indicated one end of the ultrasonic array 24a toward the other end thereof, the ultrasonic array 24a sequentially emits the respective ultrasonic beams each toward the blood vessel 20. To this end, a beam forming circuit 70 outputs respective commands to respective pulsers 72 (only one pulser 72 is shown in FIG. 3) so that the pulsers 72 supply respective drive signals to the grouped ultrasonic transducers E and accordingly the transducers E emit the respective one-period ultrasonic waves or pulses. Each time the ultrasonic array 24a emits respective ultrasonic waves or pulses, the array 24a receives reflection waves or pulses reflected from an interface of the wall of the blood vessel 20 where acoustic impedance significantly changes, and the thus received reflection pulses or signals are supplied to the control device 32 via a multiplexer (MUX) 74; respective selector switches (SW) 76 that are for preventing returning of the ultrasonic pulses emitted; respective TGC receivers 78 that can adjust gains and amplify signals; respective band-pass filters (BPF) 80 that selectively pass signals whose frequency is equal to the frequency (10 MHz) of the ultrasonic reflection waves; respective analog-to-digital (A/D) converters 82; and the beam forming circuit 70. The ultrasonic-wave control circuit 38 has a predetermined number of pulsers 72, the same number of selector switches 76, the same number of TGC receivers 78, the same number of band-pass filters 80, and the same number of A/D converters 82, although not shown in FIG. 3. The predetermined number is equal to a number of the channels used to emit, in the beam forming operation, the respective ultrasonic waves or pulses whose phases differ from each other, that is, equal to the above-indicated predetermined number of the grouped transducers E that are simultaneously driven to generate the respective ultrasonic waves. The beam forming circuit 70 has a buffer function and a calculator function, and processes the respective reflection signals received by the ultrasonic transducers E, into a special reflection signal, e.g., a reflection signal that can be regarded as a reflection signal received by the middle one of the grouped ultrasonic transducers E as seen in the direction of extension of the ultrasonic array 24a. While the ultrasonic array 24a sequentially generates the respective ultrasonic beams toward the blood vessel 20, the beam forming circuit 70 sequentially outputs the respective special reflection signals SR. The electronic control device 32 processes the series of reflection signals representing the respective ultrasonic reflection waves or pulses, and controls the image display device 34 to display the thus processed results.

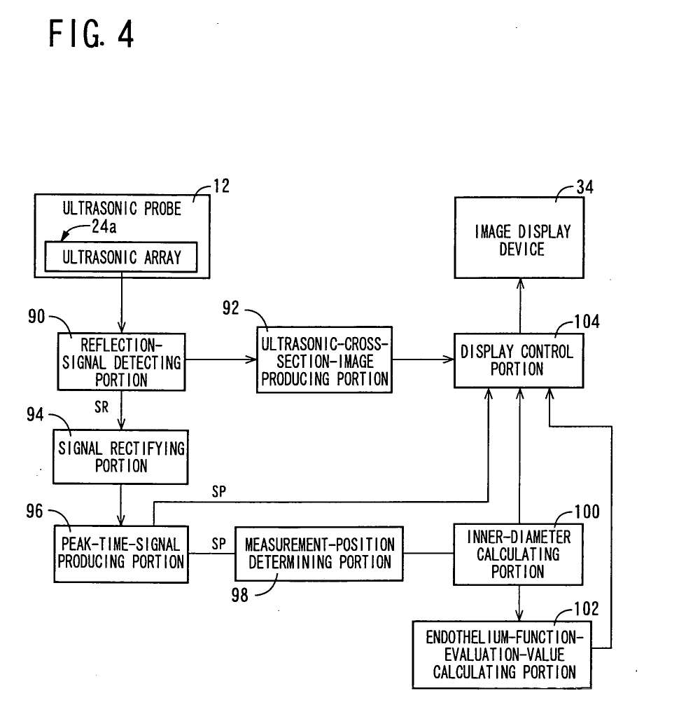

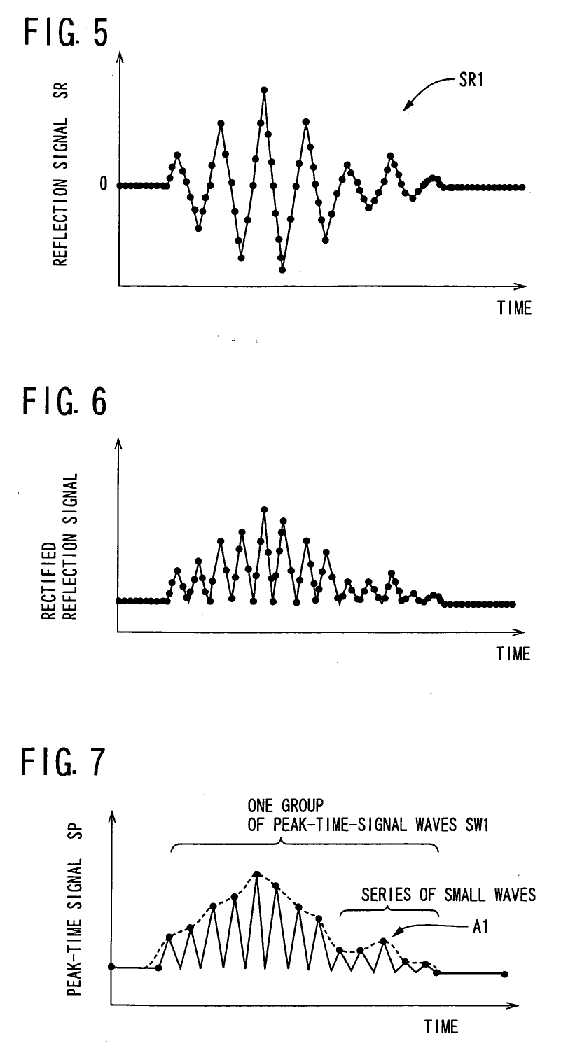

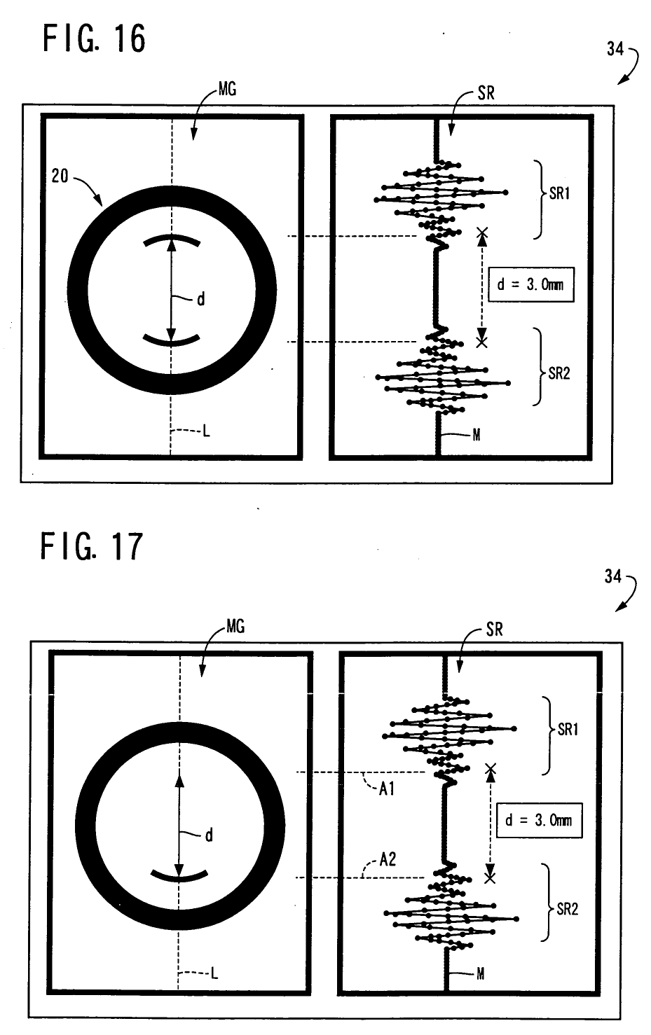

FIG. 4 is a diagrammatic view for explaining various control functions of the electronic control device 32. A reflection-signal detecting portion or means 90 detects, from the series of reflection signals received by the ultrasonic array 24a and outputted from the ultrasonic-wave control circuit 38, a reflection signal, SR, reflected from around a middle portion of the blood vessel 20, and stores the thus detected reflection signal SR in an appropriate memory, e.g., a RAM. The detected reflection signal SR consists of digital signals sampled at a frequency of, e.g., 100 MHz, and has a waveform consisting of data points (i.e., RF signal) as shown in FIG. 5. One period of the reflection signal SR, shown in FIG. 5, corresponds to the frequency (10 MHz) of the ultrasonic waves generated by the ultrasonic array 24a. Although FIG. 5 shows only one group of reflection waves, SR1, of the reflection signal SR, the reflection signal SR reflected from around the middle portion of the blood vessel 20 should contain two groups of reflection waves SR1, SR2 (see FIG. 16) that are reflected from two diametrically opposed portions of the wall of the blood vessel 20. A distance or interval between the two groups of reflection waves SR1, SR2, or between respective maximum amplitudes of the two groups of reflection waves SR1, SR2 is utilized in a manner described later.

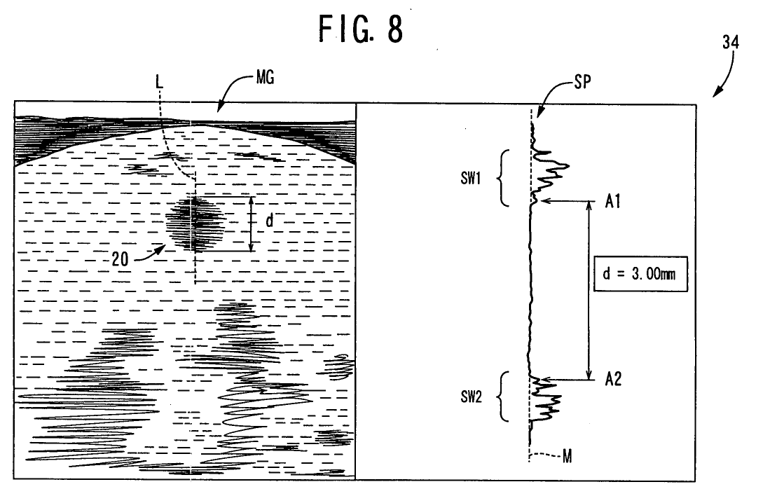

An ultrasonic-cross-section-image producing portion or means 92, well known in the art, first converts the ultrasonic reflection signal SR by rectifying and squaring the same, into the power of the same, subsequently converts the power of the signal by smoothing the same in an envelope-smoothing process, into a smoothed signal that smoothly changes, then converts the smoothed signal into a stepwise variable density signal, and finally produces a two-dimensional ultrasonic cross-section image, MG (see FIG. 8), represented by the variable density signal.

A signal detecting or rectifying portion or means 94 carries out a wave detection, i.e., a full-wave rectification in which negative portions of the reflection signal SR, shown in FIG. 5, are changed to be positive, and thereby converts the reflection signal SR into the rectified reflection signal having an absolute-value waveform as shown in FIG. 6. A peak-time-signal producing portion or means 96 produces a peak-time signal, SP, consisting of data points each of which represents a magnitude and a time position (i.e., a time of occurrence) of a peak of a corresponding one of the reflection waves of the reflection signal SR that have been rectified by the signal rectifying portion 94. FIG. 7 shows the peak-time signal SP consisting of data points whose total number is not more than one fifth of the total number of the data points of the rectified reflection signal SR shown in FIG. 6. More specifically described, respective upper peaks of the peak-time-signal waves SP correspond to the data points, and each of respective lower peaks of the same are located between a corresponding pair of upper peaks and on a base line. The respective waveforms of the peak-time-signal waves SP may be replaced with an envelope of the upper peaks of the same, indicated by broken line in FIG. 7. The produced peak-time signal SP include two groups of peak-time-signal waves SW1, SW2 corresponding to the two groups of reflection waves (i.e., reflection-signal waves) SR1, SR2.

A measurement-position determining portion or means 98 determines, as a first measurement position, A1, a terminal point of a series of waves located in a terminal portion of a first group of peak-time-signal waves SW1 of the two groups of peak-time-signal waves SW1, SW2 corresponding to the two diametrically opposed portions of the wall of blood vessel 20, and determines, as a second measurement position, A2, an initial point of a series of waves located in an initial portion of a second group of peak-time-signal waves SW2 (see FIG. 8) of the two groups of peak-time-signal waves SW1, SW2. The measurement-position determining portion 98 determines, as an interval between the first and second groups of peak-time-signal waves SW1, SW2, an interval between the two measurement positions A1, A2 thus determined. FIG. 7 shows the first group of peak-time-signal waves SW1 corresponding to one of the two diametrically opposed portions of the wall of blood vessel 20 that is nearer to the skin 18 than the other wall portion, and additionally shows the first measurement position A1 as the terminal point of the series of waves located in the terminal portion of the first group of peak-time-signal waves SW1.

An inner-diameter calculating portion or means 100 calculates a time interval, t, between the first and second measurement positions A1, A2 determined by the measurement-position determining portion 98, and iteratively calculates, according to a pre-stored relationship between time interval t and inner diameter, d, (i.e., d=t×V), an inner diameter d of the blood vessel 20, at a predetermined timing within each of continuous heartbeat periods (e.g., a timing of occurrence of a diastolic blood pressure within each heartbeat period). In the above-indicated relationship, symbol, V, indicates a speed of sounds that travel in the living being 14, e.g., 1,530 m/sec. Thus, the inner-diameter calculating portion 100 can calculate not only a diameter d of the blood vessel 20 when the living being 14 is at rest but also a maximum diameter, dₘₐₓ, of the vessel 20 after the blood flow is resumed following the stopping thereof. However, the inner-diameter calculating portion 100 may iteratively calculates, according to the pre-stored relationship, an inner diameter d of the blood vessel 20, at a predetermined timing corresponding to a systolic blood pressure within each heartbeat period.

An endothelium-function-evaluation-value calculating portion or means 102 calculates, based on the diameter d of the blood vessel 20 when the living being 14 is at rest and the maximum diameter dₘₐₓ of the vessel 20 after the blood flow is resumed following the stopping thereof, an evaluation index, % FMD, according to a predetermined relationship, i.e., % FMD=100×(dₘₐₓ−d)/d. The evaluation index % FMD is used to evaluate the function of the endothelium of blood vessel 20 of the living being 14.

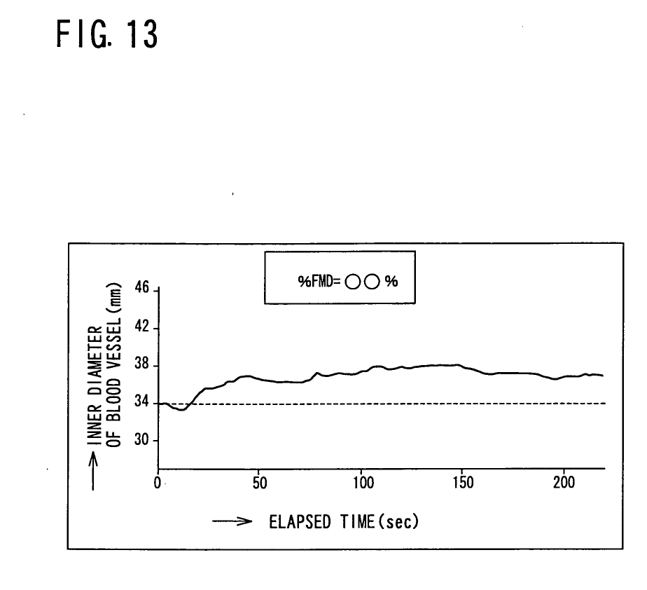

A display control portion or means 104 controls the image display device 34 to display, together with the inner diameter d calculated by the inner-diameter calculating portion 100 and the interval or range between the two measurement positions A1, A2 that corresponds to the inner diameter d, the respective waveforms of the two groups of peak-time-signal waves SW1, SW2. More specifically described, the display control portion 104 controls the image display device 34 to display the two groups of peak-time-signal waves SW1, SW2 used to calculate the inner diameter d of the blood vessel 20, and the two-dimensional ultrasonic cross-section image MG produced by the ultrasonic-cross-section-image producing portion 92, side by side, such that the two groups of peak-time-signal waves SW1, SW2 and the ultrasonic cross-section image MG can be compared with each other by an observer such as a doctor, as shown in each of FIGS. 8 through 12. In addition, the display control portion 104 controls the image display device 34 to display, at respective predetermined positions on an image screen thereof, a graph representing a time-wise change of the inner diameter values d of the blood vessel 20 that are calculated by the inner-diameter calculating portion 100 after the blood flow is resumed following the stopping thereof, and the endothelium-function evaluation index value % FMD calculated by the endothelium-function-evaluation-value calculating portion 102, as shown in FIG. 13.

FIG. 8 shows an example of an image displayed on the image screen of the image display device 34. In this example, a two-dimensional ultrasonic cross-section image MG that is produced by the ultrasonic-cross-section-image producing portion 92 and is represented by a variable-density signal, is displayed in a left-hand portion of the image screen, and a waveform defined by an envelope of a plurality of peaks of the peak-time signal SP that is produced by the peak-time-signal producing portion 96 and includes the two groups of peak-time-signal waves SW1, SW2 corresponding to the two groups of reflection waves SR1, SR2 reflected from the two diametrically opposed portions of the wall of the blood vessel 20, is displayed in a right-hand portion of the image screen, such that the image MG and the waveform are arranged side by side so as to be comparable with each other by an observer. On the image screen, the ultrasonic cross-section image MG is displayed such that a vertically downward direction corresponds to a direction of depth of the tissue as seen from the skin 18; and the waveform indicating the peak-time signal SP is displayed such that a vertically downward direction corresponds to a time axis, that is, the depth direction of the ultrasonic cross-section image MG is parallel to the time axis of the peak-time signal SP. In addition, the ultrasonic cross-section image MG is accompanied by a path, L, of the reflection signal SR along which the inner diameter d of the blood vessel 20 is measured; and the waveform indicating the peak-time signal SP is accompanied by a baseline, M, corresponding to the time axis. In addition, the two groups of peak-time-signal waves SW1, SW2 contained by the peak-time signal SP are accompanied by the measurement positions A1, A2 determined by the measurement-position determining portion 98, and the inner diameter d of the blood vessel 20 that is determined by the inner-diameter calculating portion 100 and that is represented by not only a distance corresponding to a length of an arrow, but also a digit(s). In addition, the inner diameter d of the blood vessel 20 is indicated at a position in the ultrasonic cross-section image MG that corresponds to the blood vessel 20. Moreover, the ultrasonic cross-section image MG is a so-called “B-mode” image, that is, a monochromatic variable-density image containing an inner lumen of the blood vessel 20 that has the inner diameter d indicated by an arrow.

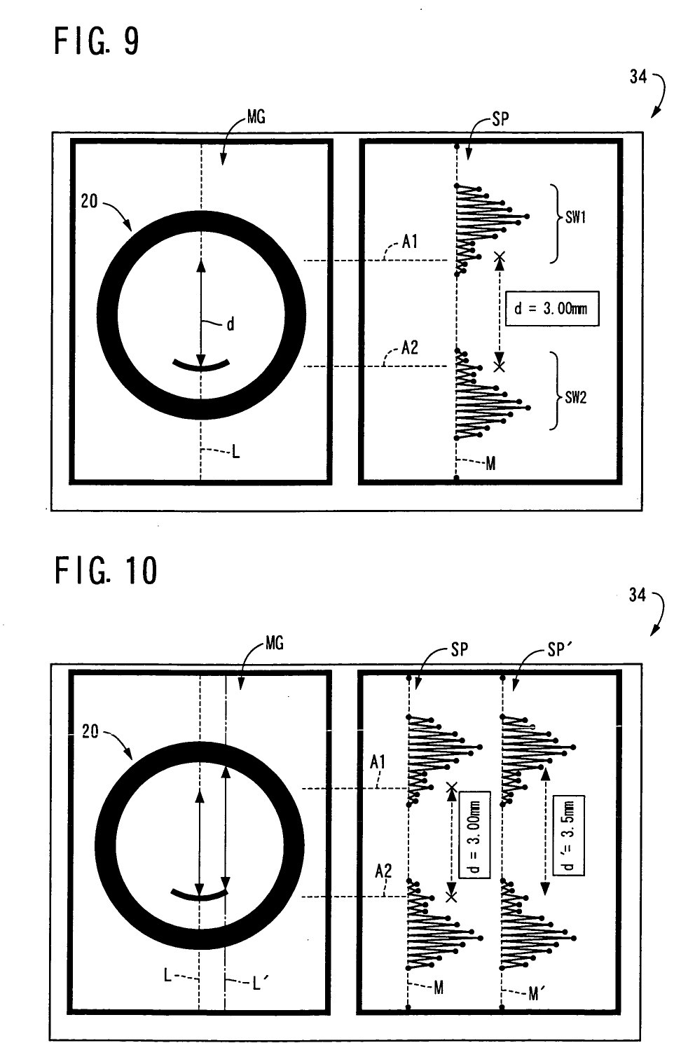

FIG. 9 shows another exemplary image displayed on the image screen of the image display device 34. Like the first example shown in FIG. 8, a two-dimensional ultrasonic cross-section image MG that is produced by the ultrasonic-cross-section-image producing portion 92 and is represented by a variable-density signal, and a waveform indicating peaks of a peak-time signal SP that is produced by the peak-time-signal producing portion 96 are displayed, side by side, on the image screen, such that the respective inner diameters d indicated by the image MG and the signal SP have a same size and have a same height position, and such that the inner diameter d of the blood vessel 20 is represented by not only a distance corresponding to a length of an arrow shown between two groups of peak-time-signal waves SW1, SW2, but also a digit(s). In addition, the ultrasonic cross-section image MG is accompanied by a path, L, (indicated by broken line) of the reflection signal SR as a basis of the peak-time signal SP; and the peak-time signal SP is accompanied by a baseline, M, thereof. In this example, the baseline M of the peak-time signal SP is converted, using the sound speed V at which sounds travel in the living being 14, from the time axis to a distance (or length) axis, such that each position on the distance axis accurately corresponds to each position on the path L of the reflection signal SR, indicated in the ultrasonic cross-section image MG. Therefore, a position on the blood vessel 20 in the ultrasonic cross-section image MG displayed in the left-hand portion of the image screen corresponds, one to one, to a position on either of the two groups of peak-time-signal waves SW1, SW2 contained by the peak-time signal SP, or a position of the arrow indicating the inner diameter d between the two groups of peak-time-signal waves SW1, SW2, each displayed in the right-hand portion of the screen image. In the ultrasonic cross-section image MG, a circular arc indicated inside the blood vessel 20 represents a portion of the endothelium of the blood vessel 20, thereby showing that the inner diameter d displayed means an inner diameter of the endothelium. On the other hand, in the ultrasonic cross-section image MG, an upper portion of the endothelium is not clearly shown. However, the first group of peak-time-signal waves SW1 corresponding to the upper portion of the endothelium contain a series of small waves corresponding to the upper portion of the endothelium. Thus, an accurate inner diameter d can be measured using the peak-time-signal waves SP. Regarding the second example shown in FIG. 9, the ultrasonic cross-section image MG is detected by inverting, and modifying (symbolizing), the black and white portions of the B-mode image (i.e., the original image). However, like the first example of FIG. 8, the ultrasonic cross-section image MG may be displayed as a B-mode image in which white portions represent strong reflection signals, that is, may be displayed as an original two-dimensional ultrasonic cross-section image per se.

FIG. 10 shows another exemplary image displayed on the image screen of the image display device 34. In this example, the image display device 34 displays the same contents as described above with respect to the second example shown in FIG. 9. In addition, the display device 34 displays, in the ultrasonic cross-section image MG, a secondary path, L′, that is parallel to the main path L of the reflection signal SR as a basis of the peak-time signal SP and is offset from the main path L passing through the central portion of the blood vessel 20; and additionally displays a peak-time signal, SP′, and a baseline, M, that correspond to the secondary path L′. In a first group of peak-time-signal waves contained by the peak-time signal SP′ corresponding to the reflection signal detected along the secondary path L′, a clearly distinguishable series of small waves corresponding to the endothelium cannot be observed. In fact, the endothelium can be recognized as just a portion of the peak-time signal SP′. Thus, an inaccurate inner diameter, d′, is measured. In the third example, an observer can recognize that an accurate inner diameter d is measured as a narrower distance between two groups of peak-time-signal waves SW1, SW2 on the baseline M than a distance between two groups of peak-time-signal waves on the baseline M′. Thus, the accuracy of measurement of inner diameter d is improved.

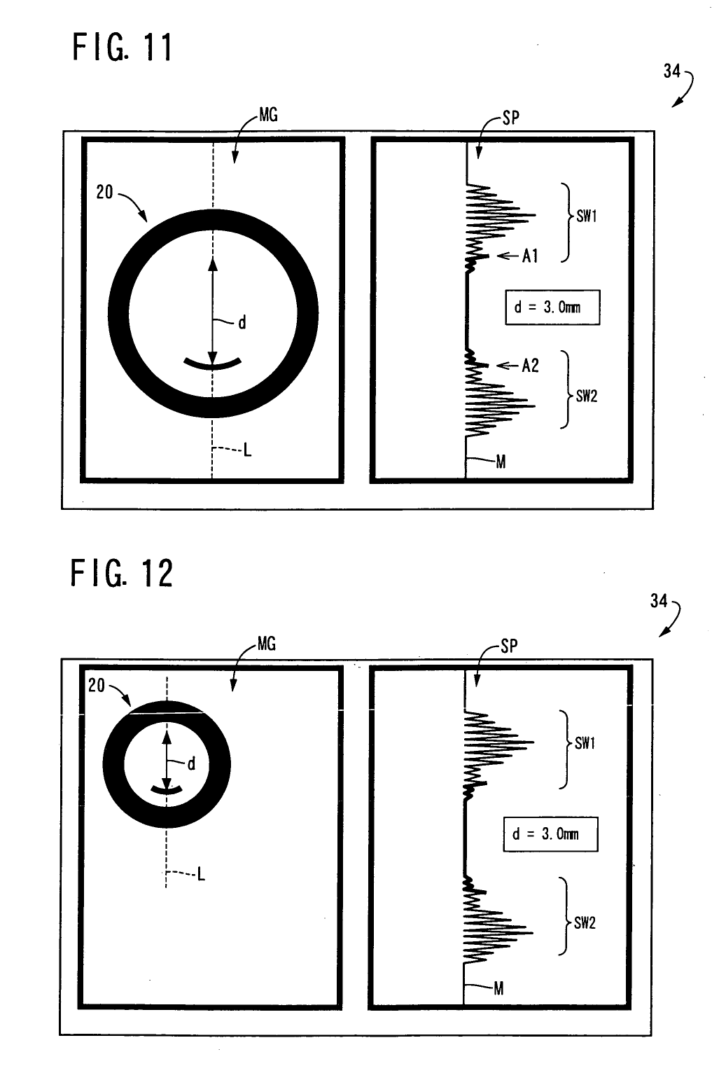

FIG. 11 shows another exemplary image displayed on the image screen of the image display device 34. In this example, the image display device 34 displays the same contents as described above with respect to the second example shown in FIG. 9, except that in the right-hand portion of the screen image where the peak-time signal SP is displayed, the arrow whose length indicates the inner diameter d in FIG. 9 is replaced with a line segment which indicates the inner diameter d and whose thickness and/or color is or are made different from a thickness and/or a color of the remaining portion of the baseline M, for easier observation purposes.

FIG. 12 shows another exemplary image displayed on the image screen of the image display device 34. In this example, an ultrasonic cross-section image MG contains a thinner or smaller blood vessel 20. In this case, if the peak-time signal SP should be displayed in such a manner that a scale on the baseline M is equal to a scale on the path L, then the peak-time signal SP would also be displayed in a small size. Hence, the peak-time signal SP is displayed in an intentionally enlarged size so as to have the same size as, e.g., the size of the peak-time signal SP shown in FIG. 11.

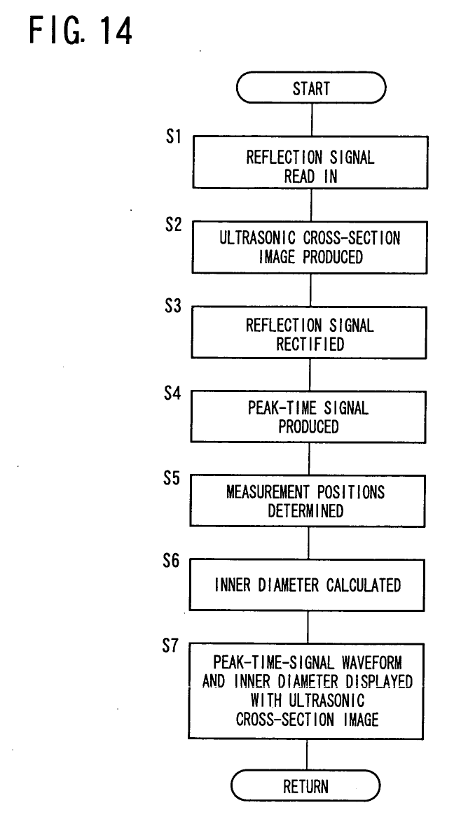

FIG. 14 is a flow chart representing a relevant portion of the control operation of the electronic control device 32. First, at Step S1 corresponding to the reflection-signal detecting portion 90, the control device 32 detects, from all the reflection signals received by the ultrasonic array 24a, the reflection signal SR reflected from the central portion of the blood vessel 20, e.g., the reflection signal SR shown in FIG. 5, and stores the thus detected reflection signal SR in an appropriate memory. Subsequently, at Step S2 corresponding to the ultrasonic-cross-section-image producing portion 92, the control device 32 first converts the reflection signal SR by rectifying and squaring the same, into the power of the same, subsequently converts the power of the signal by envelope-smoothing the same, into a smoothed signal that smoothly changes, then converts the smoothed signal into a stepwise variable density signal, and finally produces a two-dimensional ultrasonic cross-section image MG represented by the variable density signal.

Then, at Step S3 corresponding to the signal rectifying portion 94, the control device 32 carries out a wave detection, i.e., a full-wave rectification in which negative portions of the reflection signal SR, shown in FIG. 5, are changed to be positive, and thereby converts the reflection signal SR into the rectified reflection signal having the absolute-value waveform shown in FIG. 6. Subsequently, at Step S4 corresponding to the peak-time-signal producing portion 96, the control device 32 produces a peak-time signal SP, as shown in FIG. 7, that consists of data points each of which indicates a magnitude and a time position (i.e., a time of occurrence) of a peak of a corresponding one of the reflection waves SR1, SR2 that have been rectified at Step S3. The thus produced peak-time signal SP contains two groups of peak-time-signal waves SW1, SW2 corresponding to the two diametrically opposed portions of the wall of the blood vessel 20.

Then, at Step S5 corresponding to the measurement-position determining portion 98, the control device 32 determines, as a first measurement position A1, a terminal point of a series of small waves located in a terminal portion of a first group of peak-time-signal waves SW1 of the two groups of peak-time-signal waves SW1, SW2 corresponding to the two portions of the wall of the blood vessel 20, and determines, as a second measurement position A2, an initial point of a series of small waves located in an initial portion of a second group of peak-time-signal waves SW2 of the two groups of peak-time-signal waves SW1, SW2. The series of small waves located in the terminal portion of the first group of peak-time-signal, waves SW1 can be identified because they have a secondary amplitude or peak that is independent of a main amplitude or peak of the first group of peak-time-signal waves SW1 as a whole; and the series of small waves located in the initial portion of the second group of peak-time-signal waves SW2 can be identified because they have a secondary amplitude or peak that is independent of a main amplitude or peak of the second group of peak-time-signal waves SW2 as a whole. For example, the control device 32 determines, as the first measurement position A1, a remotest one of respective amplitudes of the small waves located in the terminal portion of the first group of peak-time-signal waves SW1 that are greater than a first reference amplitude, and additionally determines, as the second measurement position A2, a nearest one of respective amplitudes of the small waves located in the initial portion of the second group of peak-time-signal waves SW2 that are greater than a second reference amplitude that may, or may not, be equal to the first reference amplitude.

Then, at Step S6 corresponding to the inner-diameter calculating portion 100, the control device 32 calculates a time interval t between the first and second measurement positions A1, A2 determined at Step S5, and iteratively calculates, according to the pre-stored relationship between time interval t and inner diameter d (i.e., d=t×V), an inner diameter d of the blood vessel 20.

Finally, at Step S7 corresponding to the display control portion 104, the control device 32 controls the image display device 34 to display, together with the inner diameter value d and the range or distance between the two measurement positions A1, A2 that corresponds to the inner diameter d, the two groups of peak-time-signal waves SW1, SW2 that have been used to calculate the inner diameter d. More specifically described, as shown in each of FIGS. 8 through 12, the control device 32 controls the image display device 34 to display (a) a combination of the inner diameter d and the two groups of peak-time-signal waves SW1, SW2 used to calculate the inner diameter d, and (b) the two-dimensional ultrasonic cross-section image MG produced by the ultrasonic-cross-section-image producing portion 92 (i.e., Step S2), side by side, such that (a) the combination and (b) the cross-section image MG can be compared with each other by an observer such as a doctor.

As is apparent from the foregoing description of the illustrated embodiment, the blood-vessel-endothelium-function inspecting apparatus (i.e., the tubular-body-inner-diameter measuring apparatus) 30 detects the reflection signal SR reflected from the blood vessel (i.e., artery) 20 when the ultrasonic waves are generated from the ultrasonic probe 12 toward the blood vessel 20, determines the interval between the two groups of peak-time-signal waves SW1, SW2 corresponding to the two groups of reflection waves SR1, SR2 that are contained by the detected reflection signal SR and are respectively reflected from the two diametrically opposed portions of the wall of the blood vessel 20, and includes the inner-diameter calculating portion 100 (Step S6) that calculates, based on the determined interval, the inner diameter d of the blood vessel 20. The reflection signal SR, or the peak-time signal SP derived therefrom contains substantially all fine waves or amplitudes (i.e., strong and weak amplitudes), and accordingly contains important time-related information, that would be lost if the detected reflection signal SR is subjected to the envelope-smoothing process used to produce the ultrasonic cross-section image MG. Since the inner diameter d of the blood vessel 20 is calculated based on the interval between the two groups of reflection waves SR1, SR2 that finely or accurately correspond to the endothelium of the arterial vessel 20, the thus calculated inner diameter d can enjoy a high accuracy.

In addition, in the blood-vessel-endothelium-function inspecting apparatus 30, the peak-time-signal producing portion 96 (Step S4) produces, from the rectified reflection signal SR containing the two groups of reflection waves SR reflected from the blood vessel 20, the peak-time-signal waves SW1, SW2 each of which indicates the magnitude, and the time, of a corresponding one of the reflection waves SR1, SR2 of the two groups, and the inner-diameter calculating portion 100 (Step S6) calculates the inner diameter d of the blood vessel 20, based on the interval between the two groups of peak-time-signal waves SW1, SW2 corresponding to the two groups of reflection waves SR1, SR2 reflected from the blood vessel 20. Thus, the accurate inner diameter d can be measured. In addition, since each of the peak-time-signal waves SW1, SW2 consists of a data point indicating the magnitude, and the time, of a corresponding one of the reflection waves SR1, SR2, an amount of data as the peak-time signal SP is only from one third to one fifth of an amount of data as the sampled time-discrete reflection signal SR. This contributes to reducing the capacity of the memory or hard disc employed by the control device 32, and decreasing the signal-processing load of the same 32.

In addition, in the blood-vessel-endothelium-function inspecting apparatus 30, the measurement-position determining portion 8 (Step S5) determines the two measurement positions A1, A2 to measure the interval between the two groups of peak-time-signal waves SW1, SW2 that are contained by the peak-time signal SP, and the inner-diameter calculating portion 100 (Step S6) calculates the inner diameter d of the blood vessel 20, based on the interval (or distance) between the two measurement positions A1, A2 determined by the measurement-position determining portion 98. Since the inner diameter d of the blood vessel 20 is calculated based on the interval between the two measurement positions A1, A2 determined by the measurement-position determining portion 98, the thus calculated inner diameter d can enjoy an improved accuracy.

In addition, in the blood-vessel-endothelium-function inspecting apparatus 30, the measurement-position determining portion 98 (Step S5) determines, as the two measurement positions A1, A2 to measure the interval between the two groups of peak-time-signal waves SW1, SW2 that correspond to the two groups of reflection waves SR1, SR2 reflected from the two diametrically opposed portions of the wall of the blood vessel 20, the terminal end A1 of the first array of small waves located in the terminal portion of the first group of peak-time-signal waves SW1, and the initial end A2 of the second array of small waves located in the initial portion of the second group of peak-time-signal waves SW2. Therefore, the inner-diameter calculating portion 100 (Step S6) calculates the inner diameter d of the blood vessel 20, based on the interval between the terminal end A1 of the first array of small waves located in the terminal portion of the first group of peak-time-signal waves SW1, and the initial end A2 of the second array of small waves located in the initial portion of the second group of peak-time-signal waves SW2. Thus, a more accurate inner diameter d of the blood vessel 20 can be measured while the endothelium of the vessel 20 is taken into account.

In addition, in the blood-vessel-endothelium-function inspecting apparatus 30, the display control portion 104 (Step S7) controls the image display device 34 to display the waveform of the peak-time signal SP containing the two groups of peak-time-signal waves SW1, SW2. Therefore, an observer such as a doctor can observe, on the image display device 34, the inner diameter d of the blood vessel 20 that corresponds to the interval between the two groups of peak-time-signal waves SW1, SW2.

In addition, in the blood-vessel-endothelium-function inspecting apparatus 30, the ultrasonic-cross-section-image producing portion 92 (Step S2) produces, based on the reflection signal SR from the blood vessel (artery) 20, the cross-section image MG of the vessel 20 present under the skin 18 of the living being 14, and the display control portion 104 (Step S7) controls the image display device 34 to display the waveform of the peak-time signal SP and the cross-section image MG, side by side, such that the time axis of the peak-time signal SP and the direction of depth of the cross-section image MG are parallel to each other. Therefore, an observer can observe, on the image display device 34, the inner diameter d of the blood vessel 20 in the state in which the waveform of the peak-time signal SP and the ultrasonic cross-section image MG can be compared with each other.

In addition, in the blood-vessel-endothelium-function inspecting apparatus 30, the ultrasonic-cross-section-image producing portion 92 (Step S2) produces, based on the reflection signal SR reflected from the blood vessel (artery) 20, the cross-section image MG of the vessel 20 present under the skin 18 of the living being 14, and the display control portion 104 (Step S7) controls the image display device 34 to display the waveform of the peak-time signal SP and the cross-section image MG, such that the interval between the two groups of peak-time-signal waves SW1, SW2 contained by the peak-time signal SP (more specifically described, the interval between the terminal end of the first group of peak-time-signal waves SW1 and the initial end of the second group of peak-time-signal waves SW2), and the inner diameter d of the wall of the blood vessel 20, contained by the cross-section image MG, have a same size (i.e., a same length) and a same position with respect to the time axis and the depth direction (i.e., a vertical direction) on the image screen of the image display device 34. Therefore, an observer can observe, on the image display device 34, the interval between the two groups of peak-time-signal waves SW1, SW2 contained by the peak-time signal SP, and the inner diameter d of the blood vessel 20 contained by the cross-section image MG, in the state in which the interval and the lumen of the vessel 20 are arranged side by side so as to be comparable with each other and have the same size and the same height position. Thus, the observer can more easily observe the inner diameter d of the blood vessel 20 as the tubular body or tissue of the living being 14.

Next, there will be described a second embodiment of the present invention. The same reference numerals as used in the above-described first embodiment will be used to designate the corresponding elements or parts of the second embodiment, and the description thereof is omitted. The second embodiment is identical with the first embodiment, except for some control functions of the electronic control device 32 that will be described below.

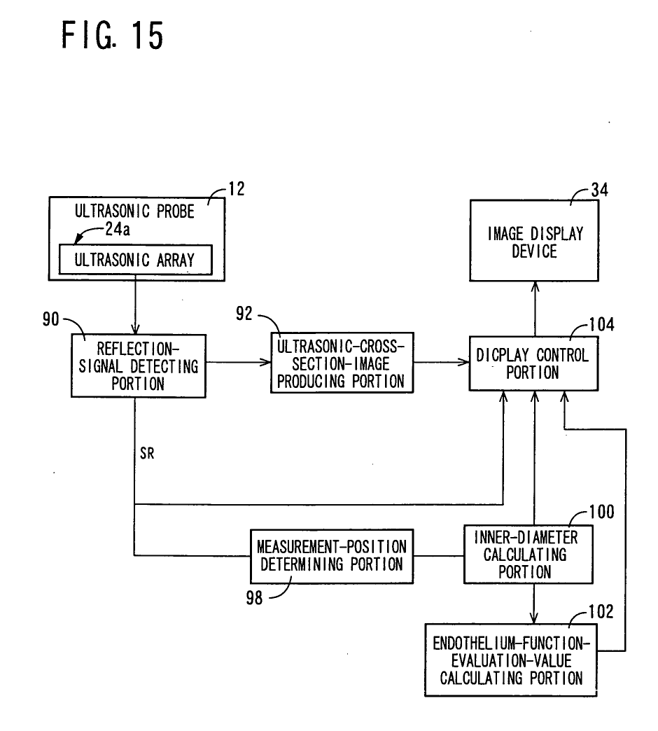

FIG. 15 is a diagrammatic view for explaining various control functions of the electronic control device 32 employed in the second embodiment. The second embodiment differs from the first embodiment in that the reflection signal SR is not converted into the peak-time signal SP and an inner diameter d of the blood vessel 20 is directly calculated based on the two groups of reflection waves SR1, SR2 contained by the reflection signal SR. In FIG. 15, a measurement-position determining portion or means 98 determines, as a first measurement position, A1, a terminal point of a series of small waves located in a terminal portion of a first group of reflection-signal waves (or reflection waves) SR1 of two groups of reflection-signal waves SR1, SR2 that are contained by the reflection signal SR (shown in FIG. 5) detected by the reflection-signal detecting portion 98 and are reflected from the two diametrically opposed portions of the wall of the blood vessel 20, and determines, as a second measurement position A2, an initial point of a series of small waves located in an initial portion of a second group of reflection-signal waves SR2 of the two groups of reflection-signal waves SR1, SR2. The measurement-position determining portion 98 determines, as an interval between the first and second groups of reflection-signal waves SR1, SR2, an interval between the two measurement positions A1, A2 thus determined. For example, the determining means 98 determines, as the first measurement position A1, a position or point where one of respective amplitudes of the small waves located in the terminal portion of the first group of reflection-signal waves SR1 exceeds a first reference amplitude, and additionally determines, as the second measurement position A2, a position or point where one of respective amplitudes of the small waves located in the initial portion of the second group of reflection-signal waves, SR2 exceeds a second reference amplitude that may, or may not, be equal to the first reference amplitude. An inner-diameter calculating portion or means 100 calculates an inner diameter d of the blood vessel 20, based on the interval between the first and second measurement positions A1, A2 determined by the measurement-position determining portion 98. A display control portion or means 104 controls the image display device 34 to display, together with the inner diameter value d calculated by the inner-diameter calculating portion 100 and the interval or range between the two measurement positions A1, A2 that corresponds to the inner diameter d, the two groups of reflection-signal waves SR1, SR2. More specifically described, the display control portion 104 controls the image display device 34 to display the two groups of reflection-signal waves SR1, SR2 used to calculate the inner diameter d of the blood vessel 20, and a two-dimensional ultrasonic cross-section image MG produced by the ultrasonic-cross-section-image producing portion 92, side by side, such that the two groups of reflection-signal waves SR1, SR2 and the ultrasonic cross-section image MG can be compared with each other by an observer such as a doctor, as shown in each of FIGS. 16 and 17 in which a waveform of the reflection signal SR (i.e., RF signal) consists of data points that are sampled at a frequency of 100 MHz.

Each of FIGS. 16 and 17 shows an example of an image displayed on the image screen of the image display device 34. Like the example shown in each of FIGS. 9 through 11, a two-dimensional ultrasonic cross-section image MG that is produced by the ultrasonic-cross-section-image producing portion 92 and is represented by a variable-density signal, and the reflection signal SR detected by the reflection-signal detecting portion 90 are displayed, side by side, on the image screen, such that the respective inner diameters d indicated by the image MG and the signal SR have a same size (i.e., a same length), and such that the inner diameter d of the blood vessel 20 is represented by not only a distance corresponding to a length of an arrow between the two groups of reflection-signal waves SR1, SR2, but also a digit(s). In addition, the ultrasonic cross-section image MG is accompanied by a path, L, (indicated by broken line) of the reflection signal SR; and the reflection signal SR is accompanied by a baseline, M, thereof. In this example, the baseline M of the reflection signal SR is converted, using a sound speed V at which sounds travel in the living being 14, from a time axis to a distance (or length) axis, such that all positions on the distance axis coincide with all positions on the path L of the reflection signal SR, indicated in the ultrasonic cross-section image MG, respectively. Therefore, positions on the blood vessel 20 in the ultrasonic cross-section image MG displayed in the left-hand portion of the image screen correspond, one to one, to positions on the two groups of reflection-signal waves SR1, SR2 contained by the reflection signal SR, or positions on the arrow that is located between the two groups of reflection-signal waves SR1, SR2 and indicates the inner diameter d, each displayed in the right-hand portion of the image screen. In the ultrasonic cross-section image MG, a circular arc indicated inside the blood vessel 20 represents a lower portion of an endothelium of the vessel 20, thereby showing that the inner diameter d means an inner diameter of the endothelium. However, in the ultrasonic cross-section image MG, an upper portion of the endothelium is not clearly shown. However, the first group of reflection-signal waves SR1 corresponding to the upper portion of the endothelium contain a series of small waves corresponding to the upper portion of the endothelium; and the second group of reflection-signal waves SR2 corresponding to the lower portion of the endothelium contain a series of small waves corresponding to the lower portion of the endothelium. Thus, an accurate inner diameter d of the blood vessel 20 can be measured using the reflection signal SR.

Regarding the first example shown in FIG. 16, a pair of circular arcs are indicated inside the blood vessel 20, and cooperate with each other to represent the endothelium of the vessel 20. On the other hand, regarding the second example shown in FIG. 17, only one circular arc is indicated inside the blood vessel 20, and it represents a portion of the endothelium of the vessel 20. That is, the circular arc corresponds to the upper portion of the endothelium of the blood vessel 20 that is located on the side of the skin 18. Even in the latter case, the first group of reflection-signal waves SR1 corresponding to the upper portion of the endothelium contain the series of small waves reflected from the upper portion of the endothelium, and the inner diameter d is calculated based on the interval between the terminal end of the series of small waves contained by the first group of reflection-signal waves SR1 and the initial end of the series of small waves contained by the second group of reflection-signal waves SR2, as displayed in the right-hand portion of the screen image. In each of FIGS. 16 and 17, the ultrasonic cross-section image MG is produced by inverting, and modifying (symbolizing), the black and white portions of the B-mode image (i.e., the original ultrasonic image). However, like the example of FIG. 8, the ultrasonic cross-section image MG may be displayed as the B-mode image, i.e., may be displayed as the original two-dimensional ultrasonic cross-section image that is represented by variable densities proportional to intensities of the reflection signal SR.

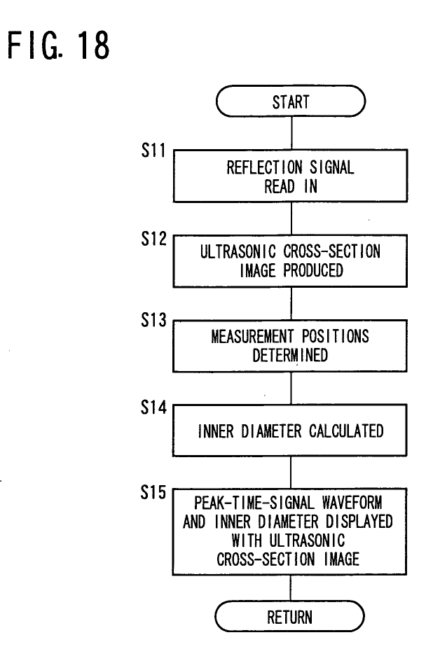

FIG. 18 is a flow chart representing a relevant portion of the control operation of the electronic control device 32 employed by the second embodiment. Step S11 corresponding to the reflection-signal detecting portion 90, and Step S12 corresponding to the ultrasonic-cross-section-image producing portion 92 are identical with Step S1 and Step S2 of FIG. 14, respectively. In short, at Step S11, the control device 32 detects, from all the reflection signals received by the ultrasonic array 24a, the reflection signal SR reflected from the central portion of the blood vessel 20, e.g., the reflection signal SR shown in FIG. 5, and stores the thus detected reflection signal SR in an appropriate memory and, at Step S2, the control device 32 first converts the reflection signal SR by rectifying and squaring the same, into the power of the same, subsequently converts the power of the signal by envelope-smoothing the same, into a smoothed signal that smoothly changes, then converts the smoothed signal into a stepwise variable density signal, and finally produces a two-dimensional ultrasonic cross-section image MG represented by the variable density signal. This two-dimensional ultrasonic cross-section image MG is a so-called B-mode image.

Then, at Step S13 corresponding to the measurement-position determining portion 98, the control device 32 determines, as a first measurement position A1, a terminal point of a series of small waves located in a terminal portion of a first group of reflection-signal waves SR1 of the two groups of reflection-signal waves SR1, SR2 that are contained by the reflection signal SR detected at Step S11 and are reflected from the two diametrically opposed portions of the wall of the blood vessel 20, and determines, as a second measurement position A2, an initial point of a series of small waves located in an initial portion of a second group of reflection-signal waves SR2 of the two groups of reflection-signal waves SR1, SR2. The series of small waves located in the terminal portion of the first group of reflection-signal waves SR1 can be identified because they have a secondary amplitude or peak that is independent of a main amplitude or peak of the first group of reflection-signal waves SR1 as a whole; and the series of small waves located in the initial portion of the second group of reflection-signal waves SR1 can be identified because they have a secondary amplitude or peak that is independent of a main amplitude or peak of the second group of reflection-signal waves SR1 as a whole. For example, the control device 32 determines, as the first measurement position A1, a remotest one from the skin 18 of respective amplitudes of the small waves located in the terminal portion of the first group of reflection-signal waves SR1 that are greater than the first reference amplitude, and additionally determines, as the second measurement position A2, a nearest one to the skin 18 of respective amplitudes of the small waves located in the initial portion of the second group of reflection-signal waves SR2 that are greater than the second reference amplitude.

Then, at Step S14 corresponding to the inner-diameter calculating portion 100, the control device 32 calculates a time interval t between the first and second measurement positions A1, A2 determined at Step S13, and iteratively calculates, according to the pre-stored relationship between time interval t and inner diameter d (i.e., d=t×V), an inner diameter d of the blood vessel 20, based on the time interval t. Finally, at Step S15 corresponding to the display control portion 104, the control device 32 controls the image display device 34 to display, together with the inner diameter value d and the range or distance between the two measurement positions A1, A2 that corresponds to the inner diameter d, the two groups of reflection-signal waves SR1, SR2 that have been used to calculate the inner diameter d. More specifically described, as shown in each of FIGS. 16 and 17, the control device 32 controls the image display device 34 to display (a) a combination of the inner diameter d and the two groups of reflection-signal waves SR1, SR2 used to calculate the inner diameter d, and (b) the two-dimensional ultrasonic cross-section image MG produced by the ultrasonic-cross-section-image producing portion 92 (i.e., Step S12), side by side, such that (a) the combination and (b) the cross-section image MG can be compared with each other by an observer such as a doctor.

As is apparent from the foregoing description of the second embodiment, the blood-vessel-endothelium-function inspecting apparatus (i.e., the tubular-body-inner-diameter measuring apparatus) 30 detects the reflection signal SR reflected from the blood vessel (i.e., artery) 20 when the ultrasonic waves are emitted from the ultrasonic probe 12 toward the blood vessel 20, determines the interval between the two groups of reflection-signal waves SR1, SR2 that are contained by the detected reflection signal SR and are respectively reflected from the two diametrically opposed portions of the wall of the blood vessel 20, and includes the inner-diameter calculating portion 100 (Step S14) that calculates, based on the determined interval, the inner diameter d of the blood vessel 20. The reflection signal SR contains substantially all fine amplitudes (i.e., strong and weak amplitudes) that would be lost if the detected reflection signal SR is subjected to the envelope-smoothing process used to produce the ultrasonic cross-section image MG. Since the inner diameter d of the blood vessel 20 is calculated based on the interval between the two groups of reflection waves SR1, SR2 that finely or accurately correspond to the endothelium of the arterial vessel 20, the thus calculated inner diameter d can enjoy a high accuracy.

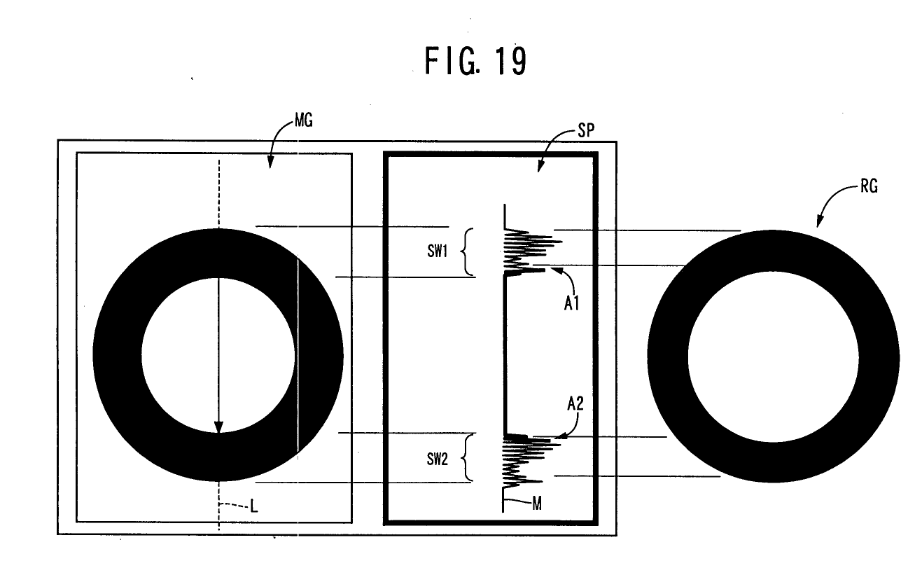

FIG. 19 shows an example of an image displayed on the image screen of the image display device 34 employed by the embodiment shown in FIG. 4, when an inner diameter of a vein or a lymph duct of the living being 14 is measured. The wall of the vein or the lymph duct is thinner than that of the artery 20, and it does not include an endothelium. Therefore, in this case, an amplitude (i.e., a rising point) that occurs to the terminal portion of the first group of peak-time-signal waves SW1 of the two groups of peak-time-signal waves SW1, SW2 contained by the peak-time signal SP and that is greater than a first reference value is determined as a first measurement position A1, and an amplitude that occurs to the initial portion of the second group of peak-time-signal waves SW2 of the two groups of peak-time-signal waves SW1, SW2 and that is greater than a second reference value is determined as a second measurement position A2. And, a time interval t between the first and second measurement positions A1, A2 is calculated, and an inner diameter d of the vein or the lymph duct is calculated based on the time interval t according to the pre-stored relationship (i.e., d=t×V). FIG. 19 additionally shows, on the right-hand side of the images displayed on the image screen, an actual cross-section image, RG, of the vein or the lymph duct that is not actually displayed on the image screen, in such a manner that the actual cross-section image RG is shown on the same scale as the scale on which the ultrasonic cross-section image MG and the peak-time signal SP are displayed on the image screen, so that a degree of accuracy of the calculated inner diameter d may be understood. As shown in FIG. 19, the ultrasonic beam is reflected at a reflection point on an interface where acoustic impedance significantly changes. Therefore, the reflection wave continues for a certain time duration from a start point corresponding to the position of the interface. Thus, the above-indicated two measurement positions A1, A2 may not accurately correspond to the two interfaces of the lumen (i.e., inner space) of the actual blood vessel 20. However, since the two measurement positions A1, A2 are determined at respective timings that are delayed each by a certain time, the calculated inner diameter d can enjoy a high accuracy.

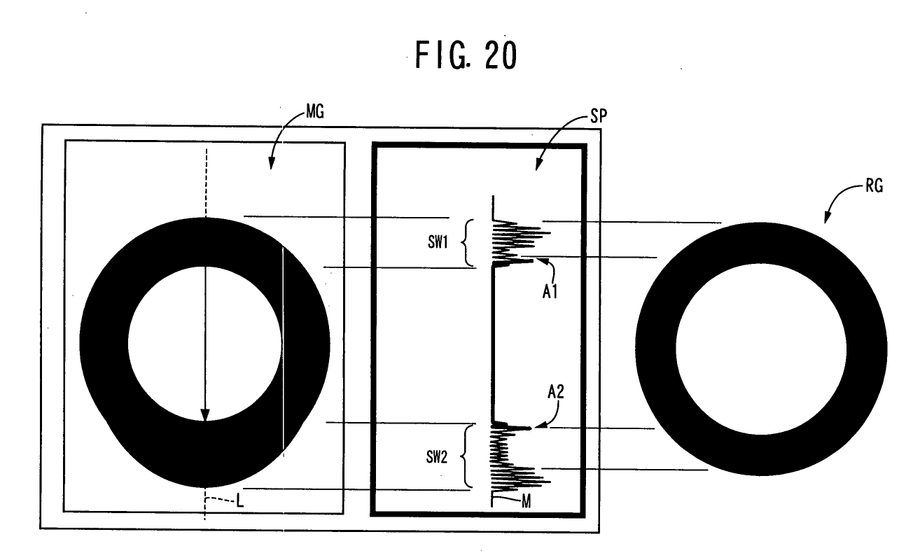

FIG. 20 shows another example of an image displayed on the image screen of the image display device 34 employed by the embodiment shown in FIG. 4, when an inner diameter of a vein or a lymph duct of the living being 14 is measured. In this case, the reflection wave continues for a longer time duration from a start point corresponding to the position of the interface remote from the skin 14. That is, the wall of the vein or the lymph duct contained by the ultrasonic cross-section image MG, displayed on the image screen, is largely offset from that of the actual cross-section image RG of the vein or lymph duct. However, in the present embodiment, an amplitude (i.e., a rising point) that occurs to the terminal portion of the first group of peak-time-signal waves SW1 of the two groups of peak-time-signal waves SW1, SW2 contained by the peak-time signal SP and that is greater than the first reference value is determined as a first measurement position A1, and an amplitude that occurs to the initial portion of the second group of peak-time-signal waves SW2 of the two groups of peak-time-signal waves SW1, SW2 and that is greater than the second reference value is determined as a second measurement position A2. And, a time interval t between the first and second measurement positions A1, A2 is calculated, and an inner diameter d of the vein or the lymph duct is calculated based on the time interval t according to the pre-stored relationship (i.e., d=t×V). Therefore, as is apparent from the comparison of the calculated inner diameter d (i.e., the interval between the two measurement positions A1, A2) with the actual cross-section image RG shown on the right-hand side of the images displayed on the image screen, the calculated inner diameter d can enjoy a high accuracy.

While the present invention has been described in its preferred embodiments, it is to be understood that the present invention may otherwise be embodied.

For example, in the first embodiment shown in FIGS. 1 through 14, the inner diameter d is calculated based on the interval between the two groups of peak-time-signal waves SW1, SW2 contained by the peak-time signal SP, shown in FIG. 7, that is detected by converting the reflection signal SR such that the peak-time-signal waves SW1, SW2 consist of the sets of data each set of which represents the magnitude, and time position, of a corresponding one of the reflection waves SR1, SR2 contained by the reflection signal SR. However, the peak-time-signal waves SW1, SW2 may be replaced with an envelope obtained by connecting, by linear interpolation or curve interpolation, the respective peak points of the peak-time-signal waves SW1, SW2. Similarly, in the second embodiment shown in FIGS. 15 through 18, the inner diameter d is calculated based on the interval between the two groups of reflection-signal waves SR1, SR2 contained by the reflection signal SR. However, the reflection-signal waves SR1, SR2 may be replaced with an envelope obtained by connecting, by linear interpolation or curve interpolation, the respective peak points of the reflection-signal waves SR1, SR2.

In addition, in each of the illustrated embodiments shown in FIGS. 1 through 20, the image display device 34 displays, on the image screen thereof, the two-dimensional ultrasonic cross-section image MG produced by the ultrasonic-cross-section-image producing portion 92 (Step S7 or Step S12), and the waveform of the peak-time signal SP or the reflection signal SR, such that the cross-section image MG and the signal waveform can be compared with each other. However, the cross-section image MG may not be displayed by the image display device 34.

Moreover, in each of the illustrated embodiments shown in FIGS. 1 through 20, the ultrasonic probe 12 is held by the sensor holding device 10 including the two link devices 48, 50. However, the sensor holding device 10 may be replaced with a different sort of sensor holding device that employs an extensible arm or a robot arm. Alternatively, the ultrasonic probe 12 may be directly worn on, e.g., the upper arm of the living being 14 with the help of, e.g., an inflatable cuff. Moreover, the ultrasonic probe 12 may be used in a state in which the probe 12 is held by a hand of an operator such as a doctor.

The present invention may be embodied with various changes and improvements that may occur to a person skilled in the art, without departing from the spirit and scope of the invention.

What is claimed is:

1. An apparatus for measuring an inner diameter of a tubular body present under a skin of a living being, by emitting an ultrasonic wave from an ultrasonic probe placed on the skin, the apparatus comprising:
a reflection-signal detecting portion which detects a reflection signal that is reflected from the tubular body when the ultrasonic wave is emitted from the ultrasonic probe; and
an inner-diameter calculating portion which calculates the inner diameter of the tubular body, based on an interval between two groups of reflection waves which are contained by the detected reflection signal and which are reflected from two diametrically opposed portions of a wall of the tubular body, respectively.

2. The apparatus according to claim 1, further comprising:
a signal rectifying portion which rectifies the detected reflection signal containing the two groups of reflection waves reflected from the tubular body; and
a peak-time-signal producing portion which produces, from the rectified reflection signal containing the two groups of reflection waves, a peak-time signal which represents a magnitude and a time position of a peak of each of respective waveforms of the reflection waves of the two groups,
wherein the inner-diameter calculating portion calculates the inner diameter of the tubular body, based on an interval between two groups of peak-time-signal waves which are contained by the produced peak-time signal and which correspond to the two groups of reflection waves, respectively.

3. The apparatus according to claim 2, further comprising a measurement-position determining portion which determines two measurement positions to measure the interval between the two groups of peak-time-signal waves which are contained by the peak-time signal and which correspond to the two groups of reflection waves, respectively, wherein the inner-diameter calculating portion calculates the inner diameter of the tubular body, based on an interval between the two measurement positions determined by the measurement-position determining portion.

4. The apparatus according to claim 3, wherein the measurement-position determining portion determines, as the two measurement positions to measure the interval between the two groups of peak-time-signal waves which are contained by the peak-time signal and which correspond to the two groups of reflection waves, respectively, a terminal end of a first series of waves located in a terminal portion of a first group of peak-time-signal waves of the two groups of peak-time-signal waves, and an initial end of a second series of waves located in an initial portion of a second group of peak-time-signal waves of the two groups of peak-time-signal waves.

5. The apparatus according to claim 2, further comprising a display control portion which controls an image display device to display a waveform of the peak-time signal containing the two groups of peak-time-signal waves.

6. The apparatus according to claim 5, further comprising an ultrasonic-cross-section-image producing portion which produces, based on the detected reflection signal reflected from the tubular body, an ultrasonic cross-section image of the tubular body present under the skin, wherein the display control portion controls the image display device to display the waveform of the peak-time signal and the ultrasonic cross-section image, side by side, such that a time axis of the waveform of the peak-time signal and a direction of depth of the cross-section image are parallel to each other.

7. The apparatus according to claim 6, wherein the display control portion controls the image display device to display the waveform of the peak-time signal and the cross-section image, such that the interval between the two groups of peak-time-signal waves contained by the peak-time signal, and the inner diameter of the wall of the tubular body, contained by the ultrasonic cross-section image, have a same size and a same position with respect to the time axis and the direction of depth.

8. The apparatus according to claim 1, further comprising a measurement-position determining portion which determines two measurement positions to measure the interval between the two groups of reflection waves which are contained by the reflection signal detected by the reflection-signal detecting portion and which are reflected from the two diametrically opposed portions of the wall of the tubular body, respectively, wherein the inner-diameter calculating portion calculates the inner diameter of the tubular body, based on an interval between the two measurement positions determined by the measurement-position determining portion.

9. The apparatus according to claim 8, wherein the measurement-position determining portion determines, as the two measurement positions to measure the interval between the two groups of reflection waves contained by the detected reflection signal reflected from the tubular body, a terminal end of a first series of waves located in a terminal portion of a first group of reflection waves of the two groups of reflection waves, and an initial end of a second series of waves located in an initial portion of a second group of reflection waves of the two groups of reflection waves.

10. The apparatus according to claim 8, further comprising a display control portion which controls an image display device to display a waveform of the detected reflection signal.

11. The apparatus according to claim 10, further comprising an ultrasonic-cross-section-image producing portion which produces, based on the detected reflection signal reflected from the tubular body, an ultrasonic cross-section image of the tubular body present under the skin, wherein the display control portion controls the image display device to display the waveform of the detected reflection signal and the ultrasonic cross-section image, side by side, such that a time axis of the waveform of the reflection signal and a direction of depth of the cross-section image are parallel to each other.

12. The apparatus according to claim 11, wherein the display control portion controls the image display device to display the waveform of the detected reflection signal and the ultrasonic cross-section image, such that the interval between the two groups of reflection waves contained by the detected reflection signal, and the inner diameter of the wall of the tubular body, contained by the cross-section image, have a same size and have a same position with respect to the time axis and the direction of depth.

13. The apparatus according to claim 1, wherein the tubular body is selected from the group consisting of an artery, a vein, and a lymph duct.
